(19) United States
(12) Patent Application Publication
(10) Pub. No.: US 2025/0243479 A1
(43) Pub. Date: Jul. 31, 2025

(54) BARCODED BEADS FOR SPATIAL ANALYSIS OF BIOMOLECULES

(71) Applicant: Stephen Macevicz, Bozeman, MT (US)

(72) Inventor: Stephen Macevicz, Bozeman, MT (US)

(21) Appl. No.: 19/032,161

(22) Filed: Jan. 20, 2025

Related U.S. Application Data
(60) Provisional application No. 63/551,689, filed on Feb. 9, 2024.
(60) Provisional application No. 63/627,418, filed on Jan. 31, 2024.

(51) Int. Cl. C12N 15/10

(52) U.S. Cl. CPC C12N 15/1065

(57) ABSTRACT

The invention is directed to methods of making spatially barcoded surfaces by formation of local mixed barcoded oligonucleotide strands and their use in spatial analysis of biomolecules.

### BACKGROUND

Analysis of spatial variations in biological processes, such as tissue-wide gene or protein expression has been enabled by the availability of surfaces with spatial barcodes comprising oligonucleotides which provide a means of indicating locations of biomolecules of interest, e.g. Stahl et al, Science, 353(6294): 78-82 (2016); Salmen et al, Nature Protocols, 13:2501-2534 (2018); Frisen et al, U.S. Pat. No. 9,593,365; and the like. In most cases, especially where high densities are required, oligonucleotide barcodes comprise random nucleotide sequences that must be determined prior to use. Thus, barcodes are typically sequenced twice: first, to determine the sequences and locations of barcodes, and second, to determine which barcodes are attached or associated with which biomolecules of interest. Vickovic et al, Nature Methods, 16(10): 987-990 (2019); Cho et al, bioRxiv (https://doi.org/10.1101/2021.01.25.427004); Chen et al Cell, 185:1777-1792 (2022); Rodriques et al, Science, 363(6434): 1463-1467 (2019); Stickels et al, Nature Biotechnology, 39(3): 313-319 (2021); and the like.

In the related field of DNA microscopy, Zhang, Weinstein and colleagues noticed that it is possible to obtain “images” of molecular distributions (for example, in a tissue) from linked barcodes in strands generated by overlap-extension PCR of selected house-keeping genes after individually barcoded strands diffuse from adjacent regions, e.g. Weinstein et al, Cell, 178:229-241 (2019). Hoffecker, Hogberg and colleagues applied the same principle to reconstruct “images” from linked barcodes formed in overlapping clusters generated by bridge PCR, e.g. Hoffecker et al, Proc. Natl. Acad. Sci., 116(39): 19282-19287 (2019). The same principle has also been applied by Fredriksson, Karlsson and colleagues to map target molecule distributions on the surfaces of single cells using physically linked rolling circle amplicons containing pairs of barcodes, e.g. International patent publications WO2022/137047 and Karlsson et al, bioRxiv doi:https://doi.org/10.1101/2023.06.05.543770 (Jun. 8, 2023). None of these studies are concerned with the use of inexpensively produced barcoded beads for increasing the efficiency of measuring spatial distributions of target nucleic acids based on the principle of geometric computation underlying the above methods.

Accordingly, fields concerned with measuring spatial distributions of target molecules, such as biomolecules, would be advanced by the availability of a cost effective spatial barcoding method which did not require the determination of barcode sequences prior to use.

### SUMMARY OF THE INVENTION

The invention is direct to methods of making and using arrays, including bead arrays, for spatial analysis of biomolecules and to kits and articles of manufacture comprising arrays, including bead arrays, made in accordance with the invention.

In some embodiments, the invention is directed to a method of making a spatially barcoded surface comprising: (a) disposing on a surface a layer of beads comprising at least a first subset of beads wherein each bead comprises first oligonucleotide strands attached, wherein the first oligonucleotide strands each comprise a barcode sequence, and a second subset of beads wherein each bead comprises second oligonucleotide strands cleavably attached, wherein the second oligonucleotide strands each comprise a barcode sequence; and (b) cleaving the second oligonucleotide strands under conditions that permit ligation of released second oligonucleotide strands to at least one first oligonucleotide strand of an adjacent bead of the first subset of the layer. In some embodiments, the above method further comprising ligating at least one of the released second oligonucleotide strands to at least one of the first oligonucleotide strands of at least one of the adjacent beads to form a mixed barcode strand.

In some embodiments, the invention comprises a method of making a spatially barcoded surface, comprising: (a) providing a surface comprising capture oligonucleotides attached thereto and a plurality of generator beads disposed thereon, wherein each bead comprises barcode oligonucleotides each comprising a barcode sequence; and (b) generating copies of said barcode oligonucleotides of the generator beads under conditions that copies of the barcode oligonucleotides from at least two different generator beads are ligated to the same capture oligonucleotide. In some embodiments of such method said conditions comprise a concentration of helper oligonucleotides effective for ligating at least a plurality of the generated copies of the barcode oligonucleotides to at least one said capture oligonucleotide.

In another aspect, methods of the invention for making a spatially barcoded surface comprise (a) disposing on a surface a layer of beads comprising oligonucleotide strands, wherein the oligonucleotide strands of each comprise a barcode sequence; and (b) releasing the oligonucleotide strands under conditions that permit the released oligonucleotide strands to be concatenated with at least one other oligonucleotide strand of an adjacent bead to form a mixed barcode strand. In some embodiments the releasing comprises replicating the first oligonucleotide strands.

In some embodiments, the invention comprises a method of making a spatially barcoded surface comprising: (a) disposing on a surface a layer of beads comprising at least a subset of beads wherein each bead comprises first oligonucleotide strands and second oligonucleotide strands, wherein the first and second oligonucleotide strands each comprise a barcode sequence such that first and second oligonucleotides of of the same bead comprise the same barcode sequence; and (b) cleaving the second oligonucleotide strands under conditions that permit ligation of released second oligonucleotide strands to first oligonucleotide strands of adjacent beads of the layer. In some embodiments, such method further includes ligating at least one of the released second oligonucleotide strands to at least one of the first oligonucleotide strands of at least one of the adjacent beads to form a mixed barcode strand.

In other embodiments, first and second oligonucleotide strands are attached to separate beads and the method is implemented by the steps: (a) disposing on a surface a layer of beads comprising at least a first subset of beads wherein each bead comprises first oligonucleotide strands attached, wherein the first oligonucleotide strands each comprise a barcode sequence, and a second subset of beads wherein each bead comprises second oligonucleotide strands cleavably attached, wherein the second oligonucleotide strands each comprise a barcode sequence; and (b) cleaving the second oligonucleotide strands under conditions that permit ligation of released second oligonucleotide strands to at least one first oligonucleotide strand of an adjacent bead of the first subset of the layer. In some embodiments, such method further comprises ligating at least one of the released second oligonucleotide strands to at least one of the first oligonucleotide strands of at least one of the adjacent beads to form a mixed barcode strand.

In other embodiments, the invention comprises methods for measuring tissue-wide expression of biomolecules comprising the steps of: (a) providing a bead array comprising beads comprising mixed barcode strands; (b) disposing a tissue slice on the bead array; (c) capturing target nucleic acids released from tissue slice by mixed barcode strands; (d) synthesizing cDNAs having mixed barcode strands from the captured target nucleic acids; (e) sequencing the cDNAs: and (f) determining relative positions of the captured target nucleic acids in the bead array from sequences of the mixed barcode strands.

In further embodiments, the invention comprises kits for implementing such methods of measuring tissue-wide expression of biomolecules, comprising a solid support comprising a layer of beads comprising mixed barcode strands.

In other embodiments, the invention comprises articles of manufacture for measuring spatial distributions of biomolecules comprising a solid support comprising a layer of beads comprising mixed barcode strands.

The invention advances the art of spatial analysis of biomolecules by reducing the amount of DNA sequence determination required to correlate barcode identities with spatial locations, thereby reducing the resources and labor required for such measurements.

### BRIEF DESCRIPTIONS OF THE DRAWINGS

FIGS. 1A-1H illustrate an embodiment of the invention wherein beads comprise first and second oligonucleotide strands releasably attached by orthogonal cleavage chemistries, and wherein the second oligonucleotide strand is cleaved so that released second oligonucleotide strands can diffuse to adjacent beads where they are ligated to first oligonucleotide strands to form mixed barcode strands.

FIG. 2 is a flow chart of an algorithm for determining relative positions of mixed barcode strands on a surface.

FIGS. 3A-3M illustrate another embodiment of the invention in which first oligonucleotide strands and second oligonucleotide strands are attached to separate beads.

FIGS. 3N-3O illustrate an embodiment of the invention in which a surface with capture oligonucleotides is provided which capture barcode oligonucleotides generated by adjacent generator beads.

FIGS. 4A-4B illustrate the use of a gel layer over beads on a surface to facilitate the delivery of reagents without disturbing bead positions.

FIGS. 5A-5B illustrate an embodiment for measuring messenger RNAs and proteins from a tissue section.

FIG. 6 illustrates an embodiment of the invention for measuring spatial distributions of messenger RNA expression.

FIGS. 7A-7B illustrate steps in the formation of DNA nanoballs (DNBs) and their deposition onto a solid surface with discrete reaction sites comprising chemical groups that preferentially bind DNBs.

FIGS. 8A-8C diagrammatically show elements of a DNB for generating secondary barcodes using SDA and polymerase arrest by a triplex structure.

FIG. 9A-9B diagrammatically show elements of a DNB for generating secondary barcodes using SDA of three-nucleotide barcodes terminated by a fourth nucleotide comprising a reversible 3′-OH terminator.

FIG. 10 shown a DNB array made in accordance with the invention and its use to measure the distribution of mRNAs in a tissue sample.

### DETAILED DESCRIPTION OF THE INVENTION

The practice of the present invention may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation and use of synthetic nucleotides, polynucleotides, molecular conjugation, surface chemistries, and the like. Specific illustrations of suitable techniques can be had by reference to the example herein below. However, other equivalent conventional procedures can also be used. Such conventional techniques, materials and descriptions can be found in standard laboratory manuals including, but not limited to, Genome Analysis: A Laboratory Manual Series (Vols. I-IV); PCR Primer: A Laboratory Manual; Retroviruses; and Molecular Cloning: A Laboratory Manual (all from Cold Spring Harbor Laboratory Press); Moore et al, Building Scientific Apparatus, Third Edition (Perseus Books, Cambridge, MA); Hermanson, Bioconjugate Techniques, 3rd Edition (Academic Press, 2013); and like references.

The invention is directed to methods, compositions, kits, articles of manufacture, and systems for producing and using spatially barcoded surfaces comprising oligonucleotide strands comprising mixed barcode sequences. In some embodiments such surfaces comprise beads with mixed barcode sequences, wherein the barcodes making up each mixed barcode sequence are from adjacent beads. In some embodiments, such surfaces comprise capture oligonucleotides thereon that capture more than one barcode oligonucleotides released or generated from adjacent generator beads thereon to form mixed barcode sequences. In some embodiments, mixed barcode sequences are produced on a bead by the capture of released barcode sequences from adjacent beads. In some embodiments, mixed barcode sequences are produced on a bead by the capture and ligation of released barcode sequences from adjacent beads. In some embodiments, a mixed barcode sequence is a single oligonucleotide strand comprising two or more barcode sequences, wherein at least one of such barcode sequences is from an adjacent bead of a bead array. The term “mixed barcode sequence” is sometimes referred to herein as a “mixed barcode strand.” One of ordinary skill in the art would understand that in many embodiments described below, steps for removing reactants, for example, by washing steps, after indicated synthesizing or ligating steps may be beneficial. For example, in steps of ligating released barcode oligonucleotide to capture oligonucleotides may benefit from washing steps to remove undesired ligation side products, such as, self-ligated oligonucleotides, barcode oligonucleotides mis-ligated to each other without being covalently bound to a surface through a capture oligonucleotide, and like side products. One of ordinary skill in the art also would understand that the methods described herein may be carried out in a wide variety of well-known apparatus, such as, for example, flow cells, flow chambers, cuvettes, or the like, such as, described in the following references which are incorporated herein by reference: Kamentsky et al, Cytometry, 12:381-387 (1991); U.S. Pat. No. 11,892,080; International Patent Publication WO2019/028047; U.S. Pat. Nos. 8,921,073; 8,173,080; 8,900,828; or the like.

As used herein, the term “spatial barcode” means a molecular indicator from which a position on a surface may be determined or indicated. A mixed barcode sequence is a spatial barcode. In some embodiments, such molecular indicators comprise surface location information encoded in a polymer sequence, such as, an oligonucleotide sequence. As mentioned above, mixed barcode sequences of the invention are derived from barcodes from adjacent beads disposed on a surface. In some embodiments, substantially every bead of a bead array comprises a unique barcode sequence, or every different bead comprises a barcode having a different sequence.

The term “bead” denotes a discrete support typically used or manipulated in populations, but which may be separated from other beads. The term is used synonymously with terms, such as, “particle,” “microparticle,” “microbead,” “microsphere,” “nanoparticle,” “DNA nanoball,” and the like. In some embodiments, the term “beads” refers to a monodisperse population of approximately spherical particles typically having diameters with a coefficient of variation less than 25 percent, or in some embodiments, less than 10 percent, or in some embodiments less than 5 percent, or in some embodiments, less than 2 percent. In some embodiments, such beads may have diameters in the range of from 1 μm to 200 μm. In some embodiments, such beads may have diameters in the range of from 1 μm to 100 μm. In other embodiments, such beads may have diameters in the range of from 2 μm to 100 μm. The composition of beads used with the invention may vary widely and in various embodiments may comprise glass, plastic, various synthetic polymers including, but not limited to, polystyrene, polysaccharide, polyethylene, DNA, and the like. In some embodiments, beads of the invention may be magnetic beads. In some embodiments, beads of the invention may be non-porous, so that synthesis or attachment of barcode oligonucleotides takes place substantially only on bead surfaces, or in other embodiments, beads may be porous, so that synthesis or attachment of barcode oligonucleotides takes place not only on beads surfaces, but also throughout the interiors of the beads. In some embodiments, porous beads comprise hydrogel beads. In other embodiments, beads may comprise DNA nanoballs; that is, rolling circle amplicons.

In some embodiments, beads on a surface are arranged in a “closely spaced” or “closely packed” array. In other words, a maximal number of beads are disposed on the surface for the area of the surface. In some embodiments, the terms mean that substantially every bead on the surface is contiguous with or touching at least a plurality of adjacent beads. In some embodiments, the terms mean that a number of beads are disposed on the surface in a density which is within ten percent of the maximal density of beads that could be disposed of the surface. In some embodiments, the terms mean that a number of beads are disposed on the surface which is within twenty-five percent of the maximal density of beads that could be disposed of the surface (assuming a monolayer of beads). In some embodiments, maximal bead density on a planar surface corresponds to a hexagonal array of beads. In some embodiments, methods and compositions of the invention comprise a surface with a diffusion inhibitor which fills the interstitial spaces between the beads, in order to control the rate of diffusion of release barcode strands from one bead to another. In some embodiments, such diffusion inhibitor may comprise a fluid, such as an oil, immiscible with a carrier fluid used to load beads onto the surface. In other embodiments, such diffusion inhibitors may comprise soluble polymers, such as agarose, poly(ethylene glycol) (PEG), dextran, poly(vinyl) alcohol, poly(vinyl) acetate, polyamide, polysaccharide, poly(lysine), polyacrylamide, poly(ethylene oxide), poly(acrylic acid), or the like. Exemplary oils include, but are not limited to, Fluoroinert-40 (FC-40); Fluoroinert-80 (FC-80); DuPont Krytox fluorinated oils; HFE-7500 (fluorinated oil); Perfluorodecalin; mineral oil; corn oil; soybean oil; silicone oil; and the like. In some embodiments, a diffusion inhibitor may be a viscosity modifier, such as, glycerol, hydroxyethyl cellulose, carboxymethyl cellulose, or the like. In some embodiments, a diffusion inhibitor may be a gel, such as a hydrogel. In some embodiments, such a hydrogel may comprise a degradable hydrogel.

In some embodiments, barcode sequences of barcode oligonucleotides may be synthesized on beads by a split-and-pool procedure, e.g. using phosphoramidite chemistry and monomers for 5′ to 3′ synthesis, such as disclosed by Macosko et al, Cell, 161:1202-1214 (2015) (supplemental materials); or using enzymatic synthesis, e.g. Godron et al, U.S. Pat. No. 11,268,091; Martin et al, U.S. patent publication US2023/0241571; or the like. Such split-and-pool procedures using all four natural nucleotides (or a subset of two or three of the four natural nucleotides in some embodiments) produces a random N-mer sequence having the form “-NNN . . . N-”, wherein the oligonucleotides synthesized on each different bead has the same random N-mer, or barcode, sequence. In some embodiments, N-mers used as barcodes in the invention have a length selected from the range of from 6 to 30 nucleotides, or from the range of from 8 to 25 nucleotides. Other types of barcode structures may be used with the method of the invention, e.g. Brenner, U.S. Pat. No. 5,635,400; Mao et al, International patent publication WO2002/097113; or the like. The number of nucleotides in the random-mer sequence determines the size of the set of barcode oligonucleotides; or, in other words, the number of different barcode sequences. In some embodiments, at least 10,000 different barcode sequences are employed, or at least 100,000 different barcode sequences are employed, or at least 500,000 different barcode sequences are employed, or at least 1,000,000 different barcode sequences are employed, or at least 10,000,000 different barcode sequences are employed. In some embodiments, the number of unique barcode sequences is greater than one million.

A wide variety of cleavable linkages may be used to releasably attached barcode oligonucleotides to beads. For example, the following references (which are incorporated by reference) disclose several suitable cleavable linkers: Leriche et al, Bioorganic & Medicinal Chemistry, 20:571-582 (2012); Urdea et al, U.S. Pat. No. 5,367,066; Monforte et al, U.S. Pat. No. 5,700,642; Glen Research application note, GR-33-11; and the like. In some embodiments, barcode oligonucleotides are releasably linked to beads by a photocleavable linkage as described in Urdea et al (cited above). In some embodiments, a photocleavable (or photo-releasable) linkage is used to attached barcode-containing oligonucleotides to beads. In some embodiments, such photocleavable linkage comprises a nitro-benzyl group as described in Urdea et al (cited above). In some embodiments, a chemically cleavable linkage is used to attach barcode oligonucleotides to beads. In some embodiments such chemically releasable linkage comprises a disulfide linkage, releasable by treatment with a reducing agent, e.g. as described in U.S. patent publication US2014/0378322 and/or Glen Research application note, GR-33-11, which are incorporated herein by reference. A surface on which beads are disposed may comprise a variety of materials, such as glass, plastic, quartz, and the like. In some embodiments, such surface is a planar surface.

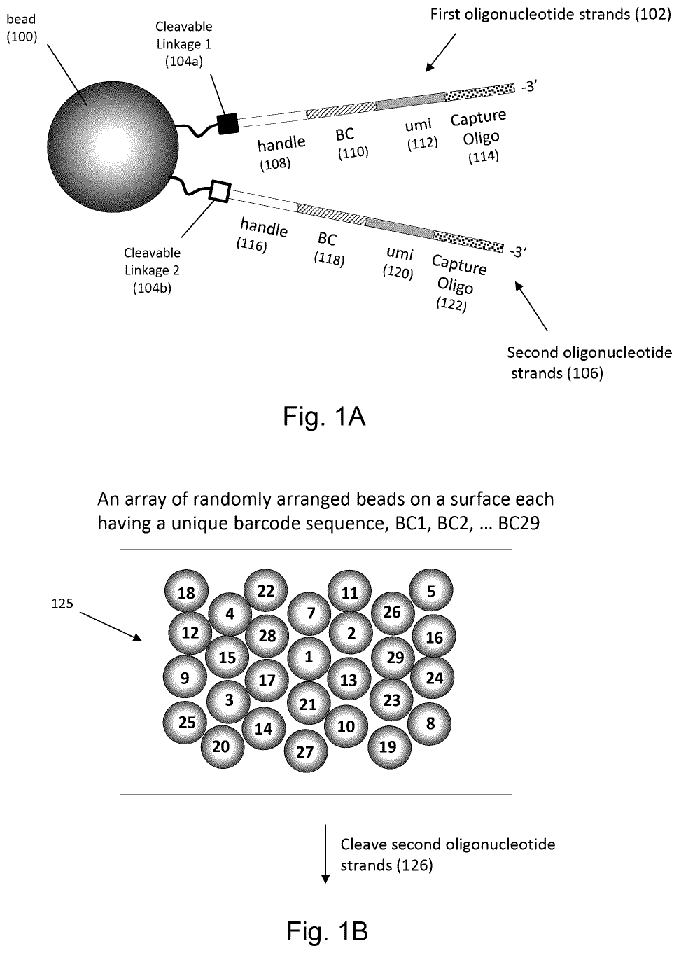

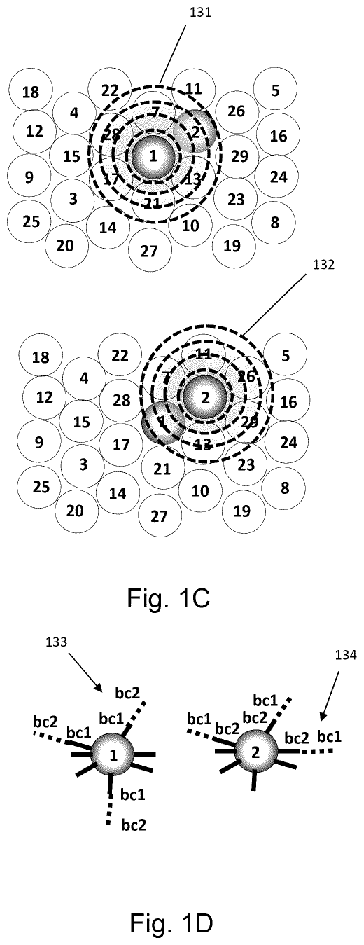

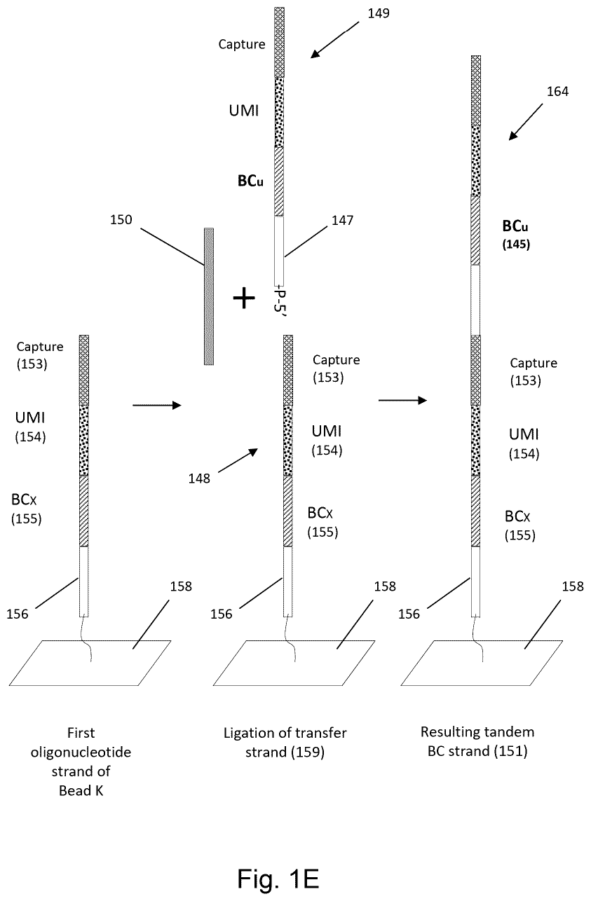

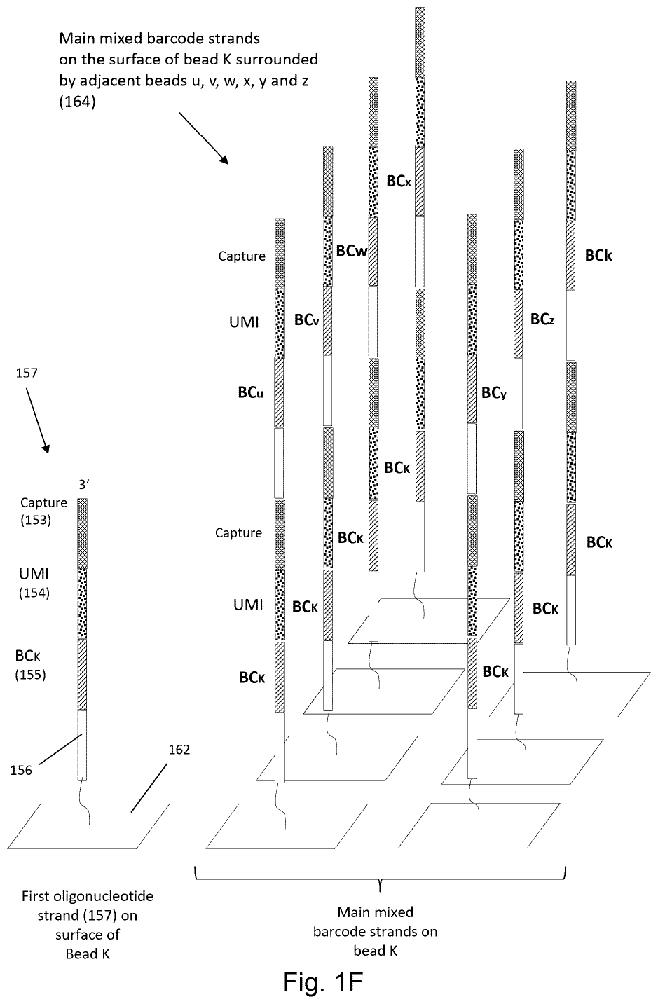

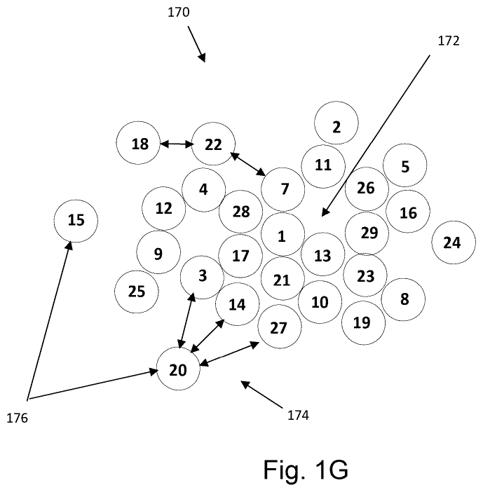

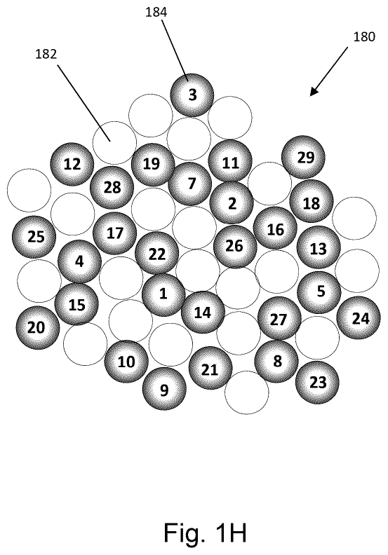

FIGS. 1A-1F illustrate an embodiment of the invention wherein each bead (100) comprises first oligonucleotide strands (102) and second oligonucleotide strands (106). First and second oligonucleotide strands (102 and 106) on the same bead each comprise a segment comprising the same barcode sequence. First and second oligonucleotide strands on different beads comprise segments with different barcode sequences. In some embodiments, first and second oligonucleotide strands (102 and 106) are each cleavably attached to beads with cleavable linkers (104a and 104b) that are cleaved using orthogonal chemistries. That is, the linkers of first oligonucleotide strands may be cleaved while leaving the linkers of the second oligonucleotide strands unaffected, and vice versa. First and second oligonucleotide strands (102 and 106) may comprise a variety of elements in addition to a segment comprising a spatial barcode sequence. Additional elements may include segments that serve as primer binding sites for amplification, segments comprising restriction endonuclease sites, segments comprising universal molecular identifiers (UMIs), and the like. FIG. 1A illustrates exemplary elements of first and second oligonucleotide strands that may be used for spatial analysis of gene expression by messenger RNA capture. First oligonucleotide strands (102) and second oligonucleotide strands (106) are attached to bead (100) by way of cleavable linkage 1 (104a) and cleavable linkage 2 (104b), respectively. From cleavable linkage 1 (104a) in a 5′-to-3′ orientation, first oligonucleotide strand (102) comprises segment (108) (referred to as a “handle” segment, barcode (110), optionally, universal molecular indicator (UMI) (112) and capture oligonucleotide (114). Likewise, in a 5′-to-3′ orientation, second oligonucleotide strand (106) comprises segment (116) referred to as a “handle” segment, barcode sequence (118), optionally, UMI (120) and capture oligonucleotide (122). Guidance for producing similar sequences attached to beads is provided by Rodriques et al, Science, 363 (6434): 1463-1467 (2019)(supplemental materials); and U.S. patent publications US20220389409; US20210123040; and the like, which references are incorporated herein by reference.

Although the segments are shown as juxtaposed segments in the figure, the indicated elements may comprise all or only a portion of the indicated segment. In other embodiments, additional segments with additional elements may be present. For a given bead, barcode sequences of the first and second oligonucleotide strands (e.g. 110 and 118) are the same. As usual, UMI sequences of each strand may be different. Handle segments (108 and 116) contain sequences (e.g. primer binding sequences) that allow manipulation (e.g. amplification, copying, ligation, or the like) of the strands using conventional techniques. Handle segments (108 and 116) for first and second oligonucleotide strands (102 and 106, respectively) may be the same or different. In some embodiments, handle segments of second oligonucleotide strands (116) each comprise a complementary region to the capture segments (114) of first oligonucleotide strands in order to facilitate ligation and formation of mixed barcode strands. Capture oligonucleotides (114 and 122) comprise sequences complementary to target nucleic acids, which may be sequences of nucleic acid analytes, such as the polyA regions of mRNAs, or artificial nucleic acids used to label molecular probes, such as antibodies. The ratio of first oligonucleotide strands to second oligonucleotide strands attached to the surface of beads may vary widely and depends on factors including, but not limited to, the bead composition, linking chemistry, size of the beads, spacing of the beads, whether diffusion inhibitors are employed, the efficiencies of the cleavable linkers, nature and concentrations of reagents used in strand ligation, and like conditions. In some embodiments, the ratio of first oligonucleotide strands to second oligonucleotide strands is 1:1; in other embodiments, the ratio is 1:2; in other embodiments, the ratio is 1:3; in other embodiments, the ratio is 2:1; in other embodiments, the ratio is 3:1.

In some embodiments, cleavable linkages 1 (104a) each comprise a photocleavable linkage, an enzymatically cleavable linkage or a chemically cleavable linkage, and cleavable linkages 2 (104b) each comprise a chemically cleavable linkage, a photocleavable linkage or an enzymatically cleavable linkage. In some embodiments, cleavable linkages 2 each comprise a photocleavable linkage. In some embodiments, such photocleavable linkage comprises a nitrobenzyl group, such as described in Urdea et al, U.S. Pat. No. 5,430,136, which is hereby incorporated by reference. In some embodiments, cleavable linkages 1 (104a) comprise an enzymatically cleavable linkage using endonuclease V in accordance with Creton, U.S. Pat. No. 11,359,221 (which is hereby incorporated by reference) or by uracil DNA glycosylase using conventional protocols.

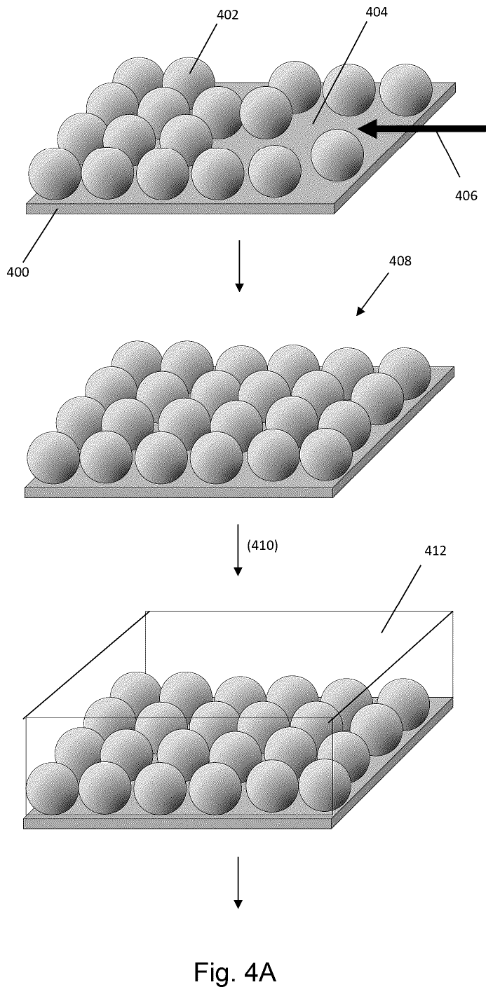

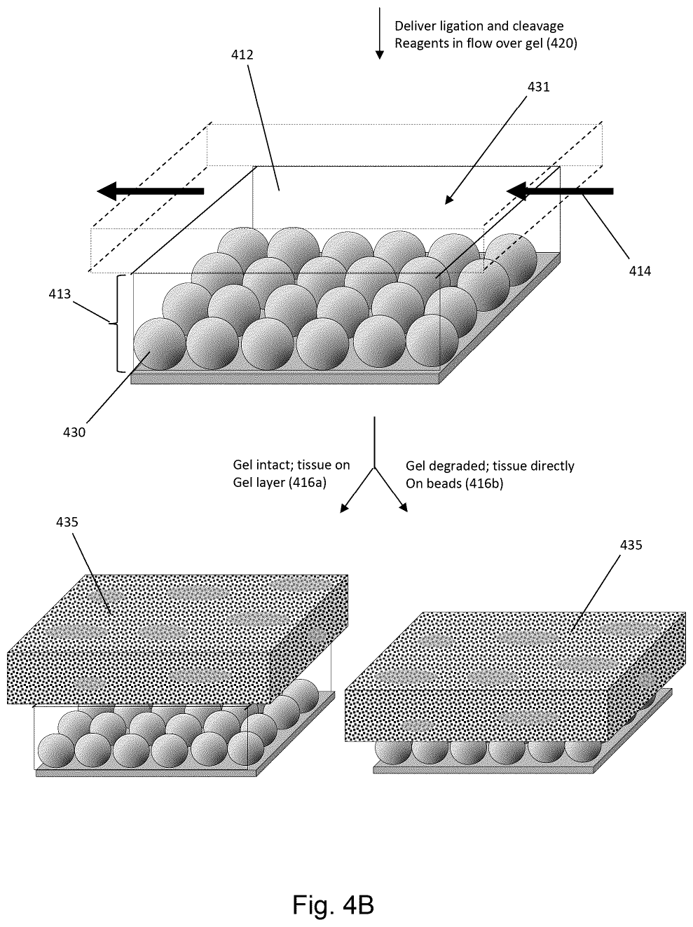

After synthesis, beads (100) with first and second oligonucleotide strands may be disposed on a surface (125) as shown in FIG. 1B. In some embodiments, beads may be immobilized or fixed to a surface using appropriate adhesives, e.g. Rodriques et al, Science, 363(6434): 1463-1467 (2019); Stickels et al, Nature Biotechnology, 39(3): 313-319 (2021); or the like. Alternatively, in some embodiments, beads may be immobilized in a hydrogel layer on the surface, wherein the hydrogel layer is permeable to selected cleavage and ligation reagents (as illustrated in FIGS. 4A and 4B). In some embodiments, such hydrogel layer may be degradable. After beads are disposed on the surface, ligation reagents are added to the surface and the second oligonucleotide strands are cleaved and allowed to diffuse to adjacent beads so that the ligation reaction illustrated in FIG. 1E takes place. In some embodiments, cleavage of cleavable linker 2 (104b) results in a released strand (149) having a 5′ phosphate to facilitate ligation released second oligonucleotide strands and first oligonucleotide strands. Ligation reagents comprise a helper oligonucleotide (e.g. 150 in FIG. 1E) which is capable of forming a duplex with capture segment (153) of attached first oligonucleotide strands (148) and handle region (147) of released second oligonucleotide strands (149), as illustrated in FIG. 1E. In some embodiments, a helper strand has a length in the range of from 10 to 50 nucleotides; or in the range of from 20 to 50 nucleotides. In some embodiments, ligation reagents further include a ligase and a ligase buffer. In some embodiments, an interval of time may take place between a cleavage step and a ligation step, wherein such an interval is defined by the times at which cleavage reagents are added to the surface and the ligation reagents are added to the surface. In some embodiments, such as illustrated in FIGS. 4A-4B, beads disposed on a surface are encased by a gel layer which prevents bulk movement of fluid at or near bead surfaces, so that diffusion of released strands is not disrupted. The interval between the steps of releasing second oligonucleotide strands and ligating released second oligonucleotide strands to first oligonucleotide strands of adjacent beads is selected to be sufficiently short that the concentration of released strands of any given bead does not reach equilibrium in the reaction volume containing all the beads on the surface. In some embodiments, the interval between cleaving and ligating is short enough so that substantially no released second oligonucleotide strands diffuse beyond immediately adjacent beads. In some embodiments, the interval between cleaving and ligating is in the range of from 10 seconds to 30 minutes. In other embodiments, the interval between cleaving and ligating is in the range of from 10 seconds to 10 minutes. One of ordinary skill would recognize that such intervals may vary greatly due to well-known factors including, but not limited to, the presence, absence or concentration of diffusion inhibitors (such as gel layers), the size of beads, the loading of second oligonucleotide strands on beads, the size of the second oligonucleotide strand, the efficiency of the cleavage of the second strands, the temperature, and the like, so that varying the above parameters to achieve objectives of particular embodiments may involve routine design choices. In some embodiments, cleavage reagents and ligation reagents are added at the same time and are permitted to react for a predetermined time, which may be in the range of from 10 seconds to 10 minutes.

After second oligonucleotide strands are cleaved a portion of the strands diffuse away from their bead of origin, as illustrated in FIG. 1C for bead with barcode 1 and bead with barcode 2. Concentric dashed circles (131 and 132) represent concentration gradients of cleaved second oligonucleotide strands that exist for an interval after cleavage. In some embodiments, second oligonucleotide strands of beads on a surface are release at substantially at the same time in the same cleavage step, with portions of the released strands diffusing to adjacent beads. FIG. 1H illustrates that in some embodiments the average distance between beads comprising barcodes (i.e. “barcoded beads”) may be adjusted by including spacer beads (182) in random bead array (180). This will have the effect of increasing the expected distance between barcode beads (e.g. 184). In some embodiments, spacer beads may have a different diameter than that of the barcoded beads. For example, spacer beads may have a smaller diameter that that of barcoded beads. In some embodiments, spacer bead diameter is in a range of from 100 percent that of barcoded beads to 25 percent that of barcoded beads. FIG. 1D illustrates resulting oligonucleotides attached to beads 1 and 2 after ligation. Mixed barcode strands (133 and 134), configured as shown, are formed on beads 1 and 2. As shown in FIG. 1D, mixed barcode strands comprise a 5′-most barcode from its bead of origin (which is sometime referred to as the “primary” barcode or barcode sequence) and the next, usually a 3′-most barcode from an adjacent bead (which is sometimes referred to herein as the “secondary” barcode or barcode sequence).

Returning to FIG. 1E, first oligonucleotide strand (157) attached to patch (158) of the surface of bead K comprises handle segment (156), barcode X (155), UMI (154) and capture segment (153). In the presence of ligation reagents after cleavage, a portion of first oligonucleotide strands will have ligated thereto second oligonucleotide strands from adjacent beads (as well as from bead K itself) to form mixed barcode strands (164) comprising barcode “BCx” (155) and barcode “BCu” (145). For a bead K surrounded by six adjacent beads (U, V, W, X, Y and Z), at least seven possible mixed barcode strands may be formed, as illustrated in FIG. 1F. Some mixed barcode oligonucleotides may comprise two or more separate or different barcode sequences. Some mixed barcode oligonucleotides may comprise two barcode sequences that may be the same or different. Some mixed barcode oligonucleotides may comprise three barcode barcode sequences that may be the same or different.

As mentioned above, in some embodiments, an image of the layer of beads may be taken to obtain data on the positions of beads on a surface (sometimes referred to herein as “image data” or “bead image data”). Such information aids in the process of associating a bead's position to that of a barcode, especially when the beads do not or cannot form a regular array (such as a hexagonal array), for example, because of different bead sizes. In FIG. 1G, a bead layer is shown that comprises an array that is only partially regular. For example, ideally, spherical beads form a regular hexagon array on a surface. However, in practice a bead array (170) may include gaps (172), irregular dispositions (176), different distances between adjacent beads (174) and the like. The configurations (or the relative distances and arrangements) of groups of beads in the optical data assists in interpreting the relative numbers of mixed barcode strands after sequencing. For example, the different distances between bead 20 and beads 3, 14 and 27 will correspond to different relative amounts of mixed barcode strands comprising (20, 3), (20, 14) and (20, 27) barcodes (where the notation “(X, Y)” represents a mixed barcode strand comprising barcode X and barcode Y). Likewise, the different distances between bead 22 and beads 18, 4 and 7 will correspond to different relative amounts of mixed barcode strands comprising (22, 18), (22, 4) and (22, 7) barcodes. Imaging a layer of beads may be carried out using a conventional optical system comprising a microscope and camera, or other recording device for collecting optical data. As used herein, the term “imaging” means collecting optical data that comprises relative positions of beads on a surface.

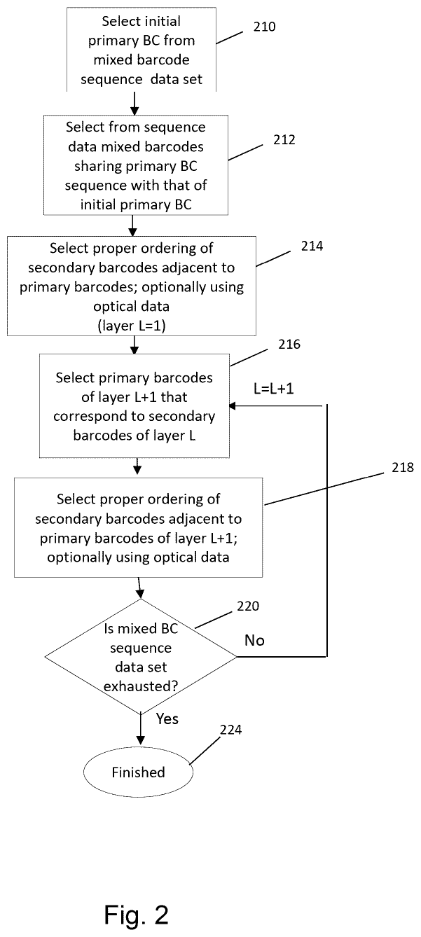

FIG. 2 contains a flow chart of an algorithm for determining the relative positions of barcodes on a surface from sequence data of mixed barcode strands. Guidance for determining relative bead positions from mixed barcode information may be found in the following references which are incorporated by reference: Bonet et al, bioRxiv: 510142 (2022.09.29); Bonet et al, Nanoscale, 15:8153 (2023); Boulgakov et al, bioRxiv: 470211 (2018.11.14); Hoffecker et al, bioRxiv: 476200 (2018.11.21); Hoffecker et al, Proc. Natl. Acad. Sci., 116 (39): 19282-19287 (2019); Boulgakov et al, Trends Biotechnol., 38(2): 154-162 (2020); Kleino et al, Comp. Struct. Biotechol. J., 20:4870-4884 (2022); Zhang et al, U.S. Pat. No. 10,655,173; Zhang et al, U.S. Pat. No. 11,339,390; and the like. In the embodiment of FIG. 2, the algorithm is based on the assumption that secondary barcodes of mixed barcode strands originate from immediately adjacent beads (to the bead from which the primary barcode originated); and such secondary barcodes are the primary barcodes of those adjacent beads. More complex and possibly more accurate algorithms may be employed; for example, algorithms that take into account relative quantities of secondary barcodes (of selected primary barcodes) in the sequence data and uses image data giving relative positions of groups of beads that may reflect the relative quantities of secondary barcodes in the sequence data. In the algorithm of FIG. 2, an initial primary barcode is selected from the sequence data (210), after which the different secondary barcodes sharing such primary barcode are each enumerated (212). The secondary barcodes indicate the primary barcodes of the adjacent beads (referred to as “layer” L in the flow chart) but do not give the ordering of such beads around the initially selected bead. To obtain the ordering (referred to herein as the “proper ordering”), the secondary barcodes of each of the primary barcodes of the adjacent beads is examined. For each such primary barcode, among its secondary barcodes will be those of its fellow adjacent beads positioned immediately next to it. Doing this for each of the primary barcodes of adjacent beads permits the adjacent beads to be placed in their proper ordering (214 and 216 in FIG. 2). This process continues (steps 216, 218 and 220) until the mixed barcode sequence data is exhausted, in which case the computation is competed (224).

In some embodiments the above-described method of the invention for manufacturing a barcoded surface area may be implemented by the following steps: (a) disposing on a surface a layer of beads comprising at least a subset of beads wherein each bead comprises first oligonucleotide strands attached and second oligonucleotide strands each cleavably attached, wherein the first and second oligonucleotide strands each comprise a barcode sequence such that first and second oligonucleotides of the same bead comprise the same barcode sequence; and (b) cleaving the second oligonucleotide strands under conditions that permit ligation of released second oligonucleotide strands to first oligonucleotide strands of adjacent beads of the layer. In some embodiments, the layer of beads on the surface is a monolayer. In some embodiments, such method further comprises ligating the released second oligonucleotide strands to the first oligonucleotide strands of the adjacent beads to form mixed barcode strands. In some embodiments, such method further comprises capturing nucleic acids from a sample with the mixed barcode strands and extending the mixed barcode strands to form mixed barcoded cDNAs. For transcriptome studies, such nucleic acids may be mRNAs. For protein studies, such nucleic acids may be oligonucleotide labels, for example, that comprise sequence codes to identify protein targets of antibodies. In some embodiments, the above method further comprises determining relative positions of the barcodes on the surface from the sequences of the barcodes of the mixed barcoded cDNAs. In some embodiments, the above method, wherein the first oligonucleotide strands are cleavably attached to the beads, further comprising cleaving and sequencing the mixed barcode cDNAs. In some embodiments, the above method further comprising imaging the layer of beads on the surface to obtain bead image data and determining positions of the barcodes on the surface from the sequences of the barcodes of the mixed barcoded cDNAs and the bead image data.

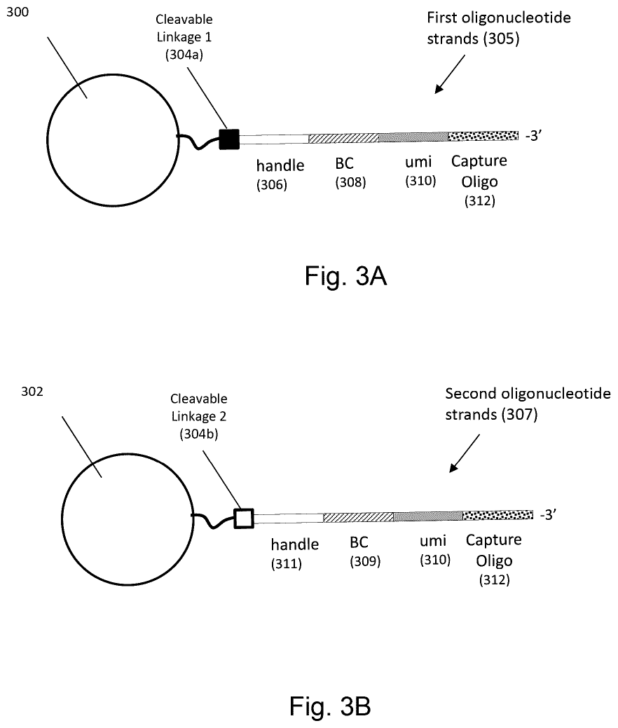

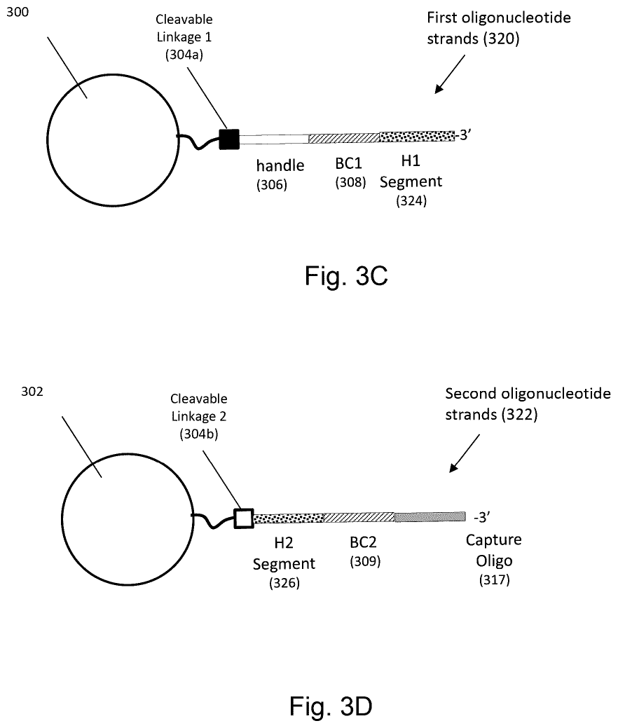

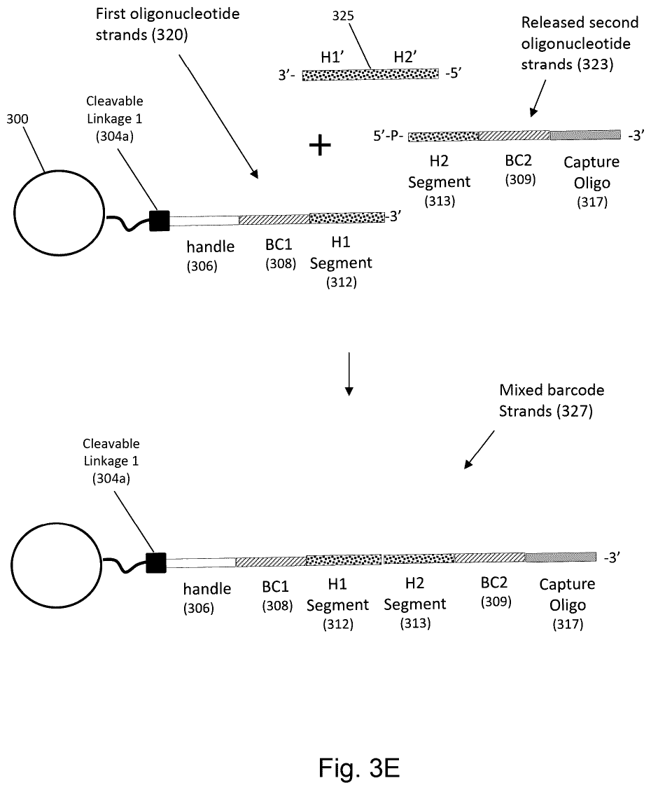

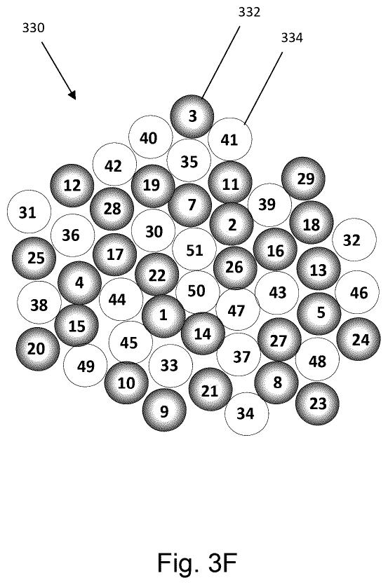

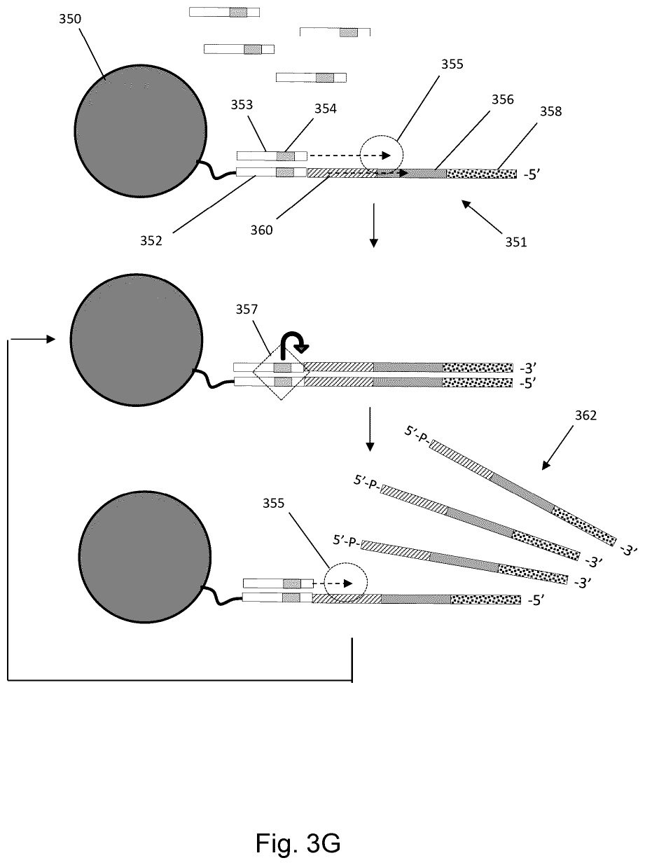

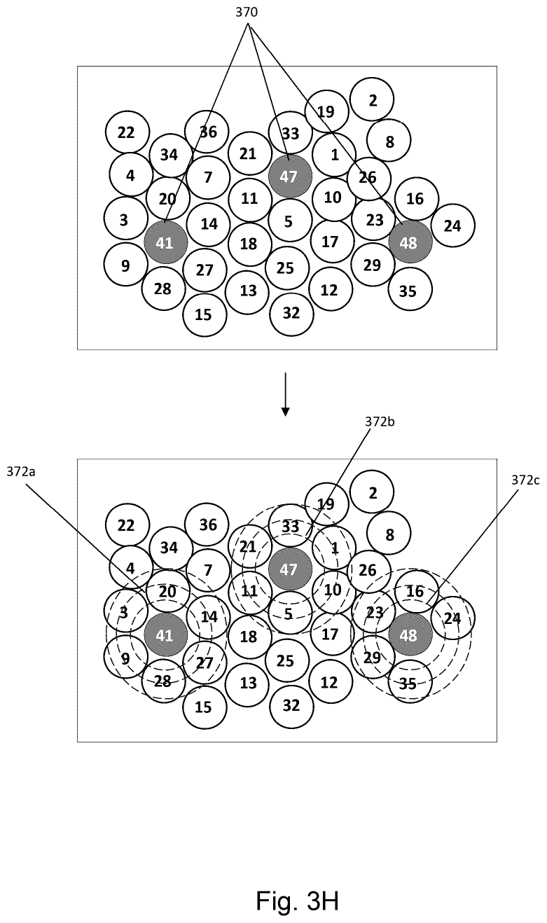

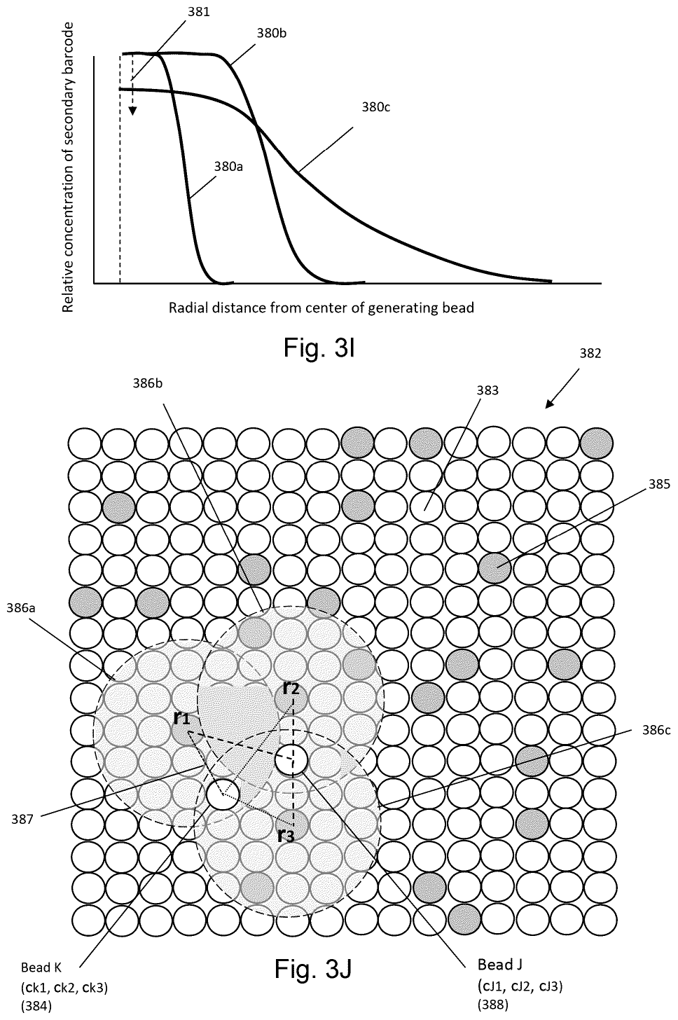

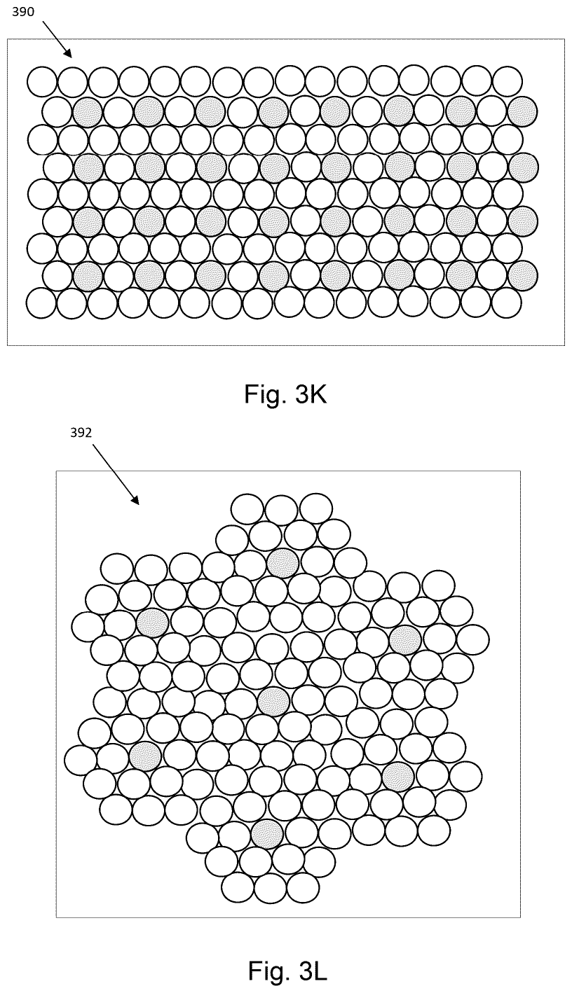

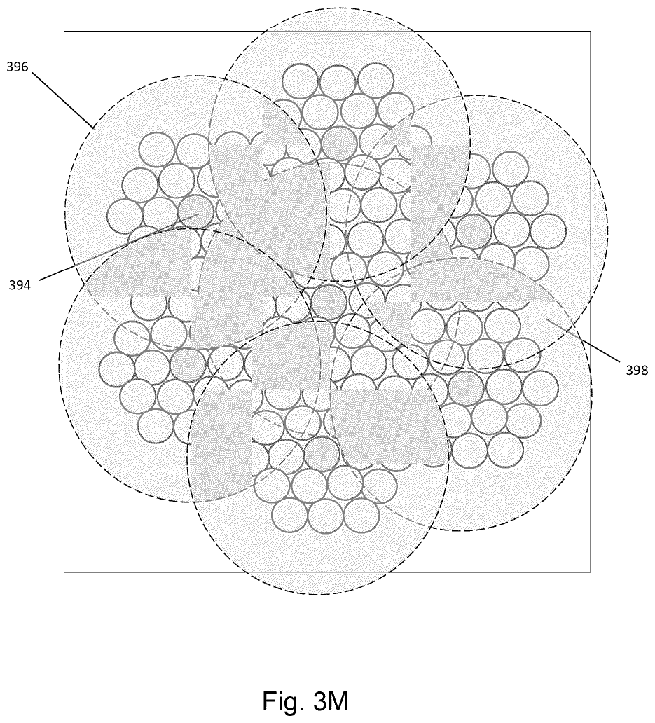

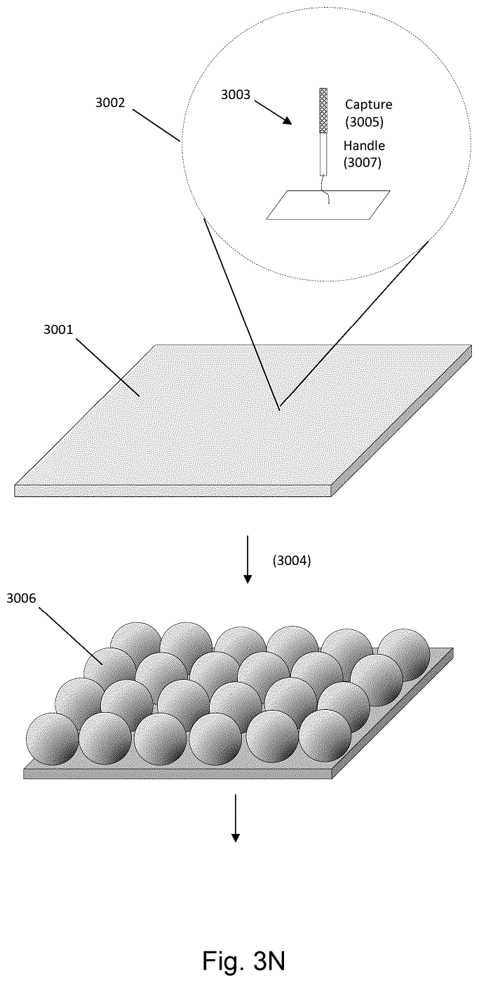

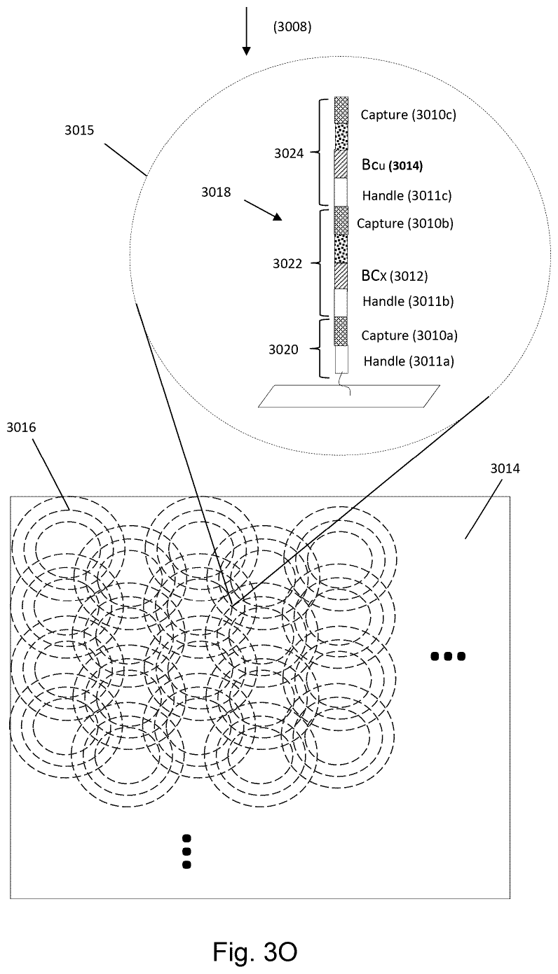

FIGS. 3A-3M illustrate another embodiment of the invention wherein the first and second oligonucleotide strands are on separate beads, for example, as shown in FIGS. 3A and 3B. There a first portion of the beads, such as bead (300), comprise only first oligonucleotide strands (305) attached by linker (304a) which may be cleavable, and a second portion of the beads, such as bead (302), comprise only second oligonucleotide strands (307) attached by cleavable linker (304b). In some embodiments, the first and second portions of the beads makes up the totality of the beads. In some embodiments, the ratio of beads with first oligonucleotide strands to beads with second oligonucleotide strands may vary in the range of from 4:1 to 1:1. In some embodiments, the ratio of beads with first oligonucleotide strands to beads with second oligonucleotide strands is in the range of from 3:1 to 1:1. As in FIG. 1A, the first and second oligonucleotide strands may comprise various elements in different segments including, but not limited to, handle sequences (306 and 311), barcodes (308 and 309), UMI sequences (310), capture sequences (312), and the like.

In some embodiments which employ first and second oligonucleotide strands attached to separate beads, first oligonucleotide strands may be provided without capture strands so that their main function is to receive released second oligonucleotide strands for ligation and to contribute a barcode to mixed barcode strands. That is, instead of a 3′-terminal capture sequence, such first oligonucleotide strands comprise a sequence primarily, or solely, designed to hybridize to and form a duplex with a helper oligonucleotide. A representative of such embodiments is illustrated in FIGS. 3C-3F. FIG. 3C shows a first oligonucleotide strand comprising handle segment (306) and barcode 1 sequence (308) as before, but in place of a capture sequence it comprises H1 segment (324) that is complementary to a 3′ end of a helper oligonucleotide. Likewise, FIG. 3D shows a second oligonucleotide strand comprising at its 5′ end (proximal to the bead) an H2 segment (326) that is complementary to a 5′ end of a helper oligonucleotide. As mentioned above, in some embodiments, cleavable linkage 2 (304b) is selected so that released second oligonucleotide strands have 5′-phosphate groups. After release of the second oligonucleotide strands and the addition of ligation reagents, the H1 and H2 segments and the helper oligonucleotides operate as shown in FIG. 3E. Helper oligonucleotides (325) hybridizes to H1 segments (312) of first oligonucleotide strands (320) and H2 segments (313) of released second oligonucleotide strands (323) to bring a 3′ end of first oligonucleotide strands into juxtaposition with a 5′-phosphate of a released second oligonucleotide strand so that ligation can occur and mixed barcode strand (327) is formed. FIG. 3F shows a random arrangement (330) of the two bead types in about a 1:1 ratio wherein shaded beads (e.g. bead 3 (332)) carry second oligonucleotide strands and unshaded beads (e.g. (334) carry first oligonucleotide strands, the latter of which solely form mixed barcode strands capable of capturing target nucleic acids. Thus, this embodiment there is a trade-off between resolution (e.g. mixed barcodes per unit surface area) and simplicity of bead synthesis (for example, in comparison to embodiments in which each bead comprises both first and second oligonucleotide strands). The different bead types may comprise different colors so that they may be distinguished in image data. Such differentiation aids in the assignment of positions to mixed barcode sequences.

FIG. 3G illustrates another embodiment wherein first and second oligonucleotide strands are initially on separate beads. In this embodiment, second oligonucleotide strands for ligation are copied or amplified from strands on a separate bead from those on which mixed barcode strands are generated or formed. FIG. 3G illustrates an embodiment based on strand displacement amplification (SDA), such as EXPAR amplification, e.g Van Ness et al, U.S. Pat. No. 7,112,432, Walker, PCR Methods and Applications, 3:1-6 (1993); Walker, U.S. Pat. No. 5,455,166 and 5648211; and the like, which are incorporated herein by reference. In some embodiments, the term “EXPAR beads” means beads configured to support EXPAR or other SDA amplification of oligonucleotides (or their complements) attached to such beads. (As in FIGS. 3A and 3B, and other figures, only a single attached oligonucleotide strand is shown for simplicity). In this embodiment, the 3′ end of template strand (351) is proximal to (and attached to) the surface of bead (350). In other embodiments, the 5′-end of template strand (351) may be attached to bead (350) (and the arrangement of elements would be reversed, for example, primer binding site (352) would be distal to bead (350)). In some embodiments, template strand (351) may comprise the following elements from its 3′ end: primer binding site (352), H2 segment (360) (described above), barcode segment (356), and complement of capture sequence (358). When primer (353) binds to primer binding site (352) a recognition site (354) for a nicking endonuclease (357) is formed, so that after extension by a strand-displacing polymerase (355), second oligonucleotide strands (362) are generated. FIG. 3H illustrates the operation of the embodiment of FIG. 3G, wherein beads 41, 47 and 48 (370) represent beads generating gradients (372a-c) of free second oligonucleotide strands, which are ligated to first oligonucleotide strands on the non-shaded beads to form mixed barcode strands. In some embodiments, beads used to generate second oligonucleotide strands may comprise a different color than beads with first oligonucleotide strands to facilitate optical identification and position determination.

Some embodiments in which first and second oligonucleotide strands are attached to separate beads may be implemented by the following steps: (a) disposing on a surface a layer of beads comprising at least a first subset of beads wherein each bead comprises first oligonucleotide strands attached, wherein the first oligonucleotide strands each comprise a barcode sequence, and a second subset of beads wherein each bead comprises second oligonucleotide strands cleavably attached, wherein the second oligonucleotide strands each comprise a barcode sequence; and (b) cleaving the second oligonucleotide strands under conditions that permit ligation of released second oligonucleotide strands to at least one first oligonucleotide strand of an adjacent bead of the first subset of the layer. In some embodiments, such method further comprises ligating at least one of the released second oligonucleotide strands to at least one of the first oligonucleotide strands of at least one of the adjacent beads to form a mixed barcode strand. In some embodiments, the method further comprises imaging the layer of beads on the surface to obtain bead image data and determining positions of the barcodes on the surface from the sequences of the barcodes of the mixed barcode strands (or copies thereof) and the bead image data. In some embodiments, the surface comprises a regular array of wells or reaction sites each configured to retain a single bead. In some embodiments, the beads are disposed randomly among the wells or reaction sites.

In some embodiments, second oligonucleotide strands are capable of being amplified and released, and the above method further comprises amplifying and releasing the second oligonucleotide strands under conditions that permit ligation of released second oligonucleotide strands to first oligonucleotide strands of at least one adjacent bead of the first subset of beads, thereby forming one or more mixed barcode strands on one or more adjacent beads. In some embodiments, such amplified, or copied, and released second oligonucleotide strands are produced by a strand displacement amplification (SDA) reaction, such as exponential amplification reaction (EXPAR).

FIGS. 3I-3M illustrate some further embodiments employing separate beads for amplifying and releasing secondary barcodes and for receiving secondary barcodes to form mixed barcode strands. FIG. 3I illustrates qualitatively a time course of concentration profiles as a function of a radial coordinate having an origin at the center of a bead on which amplifying and releasing barcode oligonucleotides, in some embodiments so-called secondary barcodes, takes place (sometimes referred to herein as “generator beads,” for example, as illustrated by gray beads (e.g. 385) in FIG. 3J). Likewise, beads configured to capture released secondary barcodes, e.g. by ligation, occasionally are referred to herein as “acceptor beads,” for example illustrated by white beads (e.g. 383) in FIG. 3J. Concentration profiles (e.g. 380a-380c) may be estimated by modeling the generation and transport of secondary barcodes (e.g. made by EXPAR) with reaction-diffusion equations. Initial profile (380a) is steep, but as time passes subsequent profiles (e.g. 380b) have a shallower slope followed by profiles, such as (380c), which show a concentration decline (381) adjacent to the surface of a generator bead and which reflect the exhaustion of precursor nucleotides for synthesizing new secondary barcodes. Of course, in some embodiments, dNTP monomers may be replenished to extend the synthesis and release of oligonucleotide strands comprising secondary barcodes. FIG. 3J illustrates a bead array wherein a substrate is patterned so that beads are arranged is a rectilinear array (for example, corresponding to a rectilinear pattern of reaction sites or wells) and wherein the beads are randomly distributed among the reaction sites or wells. In this embodiment, the effective extent of diffusing secondary barcodes is shown by disks (386a, 386b and 386c) which are associated with generator beads having centers at coordinates (r₁, r₂ and r₃, respectively). The effective extent of diffusing secondary barcodes from a particular generator bead (sometimes referred to herein as the “effective diffusion area”) means, for example, that beyond the radius of a particular disk (for example, disk (386a) of generator bead at r₁), secondary barcodes cannot be reliably measured or their concentrations are negligible, from sequencing data. An effective diffusion area depends on factors, such as, the amount of secondary barcodes synthesized and released from a particular generator bead, the presence or absence, concentration and type of diffusion inhibitor employed, temperature, efficiency of ligation chemistry used, and the like. In some embodiments, generator beads comprise oligonucleotides attached to bead surfaces and/or interiors which are capable of being replicated or amplified (either identically or as a complementary sequence) by a strand displacement amplification reaction, such as EXPAR.

FIG. 3J also shows two exemplary acceptor beads K (384) and J (388) along with dashed lines (e.g. 387) connecting the centers of beads K (384) and J (388) to the centers of generator beads at coordinates r₁, r₂ and r₃. Associated with each acceptor bead, such as, bead K and J, are values C_{K1}, C_{K2} and C_{K3} for bead K (384) and values C_{J1}, C_{J2}and C_{J3} for bead J. These values are the amounts or proportions of mixed barcode strands on beads K and J whose secondary barcodes originate from generator beads at r₁, r₂ and r₃, respectively. From such values the distances of each of the acceptor beads adjacent to the three generator beads can be determined. One of ordinary skill in the art would appreciate that, in this embodiment, the proportion of the total number of beads that are generator beads is important in determining the proportion of acceptor beads whose relative positions cannot be unequivocally determined because such beads are outside of the effective extent diffusion area of generator beads. In some implementations of this embodiment, the proportion of beads that are generator beads is equal to or greater than 10 percent; in other implementation, such proportion is in the range of from 5 to 25 percent; or in the range of from 10 to 25 percent. In some embodiments, each acceptor bead must have among its mixed barcode strands secondary barcodes from at least two different generator beads. That is, for unambiguous position determination from sequencing data, each acceptor bead must be within the effective diffusion areas of at least two generator beads.

In some embodiments, acceptor beads and generator beads may be disposed on a patterned surface in predetermined locations by employing different and orthogonal bonding chemistries (e.g. biotin-streptavidin reaction sites and wells, wells and beads of different sizes, or the like). Exemplary arrangements of generator beads (grey circles) and acceptor beads (white circles) are illustrated in FIG. 3K and 3L. Array (390) of FIG. 3K comprises 25 percent generator beads, and array (392) of FIG. 3L comprises 5.5 percent generator beads. FIG. 3M illustrates the necessary extent of disk-shaped effective diffusion areas (e.g. 396) of generator beads (e.g. 394) in array (392) of FIG. 3L for unambiguous determination of the positions of acceptor beads by their mixed barcode strand sequence data. Roughly, an acceptor bead must be within at least one overlap area, such as (398).

In some implementations of the embodiments of FIGS. 3A-3M, beads carrying second oligonucleotide strands may comprise a plurality of optically encoded beads so that each optically encoded bead comprises an associated known barcode in the second oligonucleotide strand; that is, an optically encoded bead comprises an optical label indicative of the secondary barcode released therefrom. Sets of optically encoded beads (that is, sets of beads each comprising a different optical label from a plurality of distinguishable optical labels) are well-known to those of ordinary skill in the art, e.g. Vafajoo et at, Biomed Microdevices, 20(3): 66 (2019); Yuankui et al, Chem. Soc. Rev., 44:5552 (2015); Birtwell et al, Integrative Biol., 1:345-362 (2009); Jun et al, Molecules, 17:2474-2490 (2012); and the like. In some embodiments, this barcode with a known sequence can be a secondary barcode; that is, the barcode released for ligation to a primary barcode strand. In some embodiments, such plurality is in the range of from 10 to 100; or in the range of from 25 to 100; or in the range of from 25 to 50. Preferably, the plurality is large enough so that the probability of such optically encoded beads of the same label and barcode having overlapping effective diffusion areas is low, for example, less than 5 percent. During manufacture of bead arrays, such as those of FIGS. 3J-3M, locations of optically encoded beads can be identified and defective arrays with inoperable, or less than optimal, distributions of beads carrying second oligonucleotide strands can be discarded.

FIGS. 3N-3O illustrate an embodiment wherein barcode oligonucleotides are generated (or synthesized on) and released from generator beads (e.g. each comprising a unique barcode sequence in a barcode oligonucleotide) disposed thereon, after which such barcode oligonucleotides are captured by capture oligonucleotides on the surface. In some embodiments, such capture reactions comprise helper oligonucleotides and multiple ligations of barcode oligonucleotide to the same capture oligonucleotide, thereby resulting in mixed barcode oligonucleotides on the surface. Surface (3001) in FIG. 3N comprises capture oligonucleotides (3003) shown in blow-up (3002). Typically capture oligonucleotides (3003) each comprise capture segment (3005) and handle segment (3007). Capture segment (3005) may be selected to be complementary to a portion of a helper oligonucleotide to facilitate ligation of released barcode oligonucleotides. Barcode oligonucleotides are delivered (3004) to surface (3001) by disposing generator beads (3006) on such surface. As noted above, in some embodiments one or more diffusion inhibitors may be disposed on surface (3001) along with generator beads (3006) and reagents for strand displacement reactions. After amplification and release of barcode oligonucleotides, capture reactions, and removal of the beads (3008), surface concentrations of captured barcodes will reflect the temporary concentration gradients around the generator beads, as illustrated in FIG. 3O (e.g. 3016). Oligonucleotides on the surface (3014) will comprise capture oligonucleotides comprising 0, 1, 2 or more barcode sequences, exemplified by mixed barcode oligonucleotide (3018) in blow-up (3015), which comprises original capture oligonucleotide (3020) comprising handle sequence (3011a) and capture sequence (3010a), along with ligated segment (3022) comprising handle sequence (3011b), barcode sequence BCx (3012) and capture sequence (3010b), and ligated segment (3024) comprising handle sequence (3011c), barcode sequence BCu (3014) and capture sequence (3010c). In this embodiment, a capture sequence is always always distal-most from the surface regardless of how many ligations take place. In this embodiment, in each case a helper oligonucleotide is complementary to a portion of the handle sequence and a portion of the capture sequence. As used herein sometimes, the term “capture oligonucleotide” encompasses a capture oligonucleotide on a surface which has not captured (or been ligated to) a released barcode oligonucleotide as well as a capture oligonucleotide on a surface which has captured, and been ligated to, one or more released barcode oligonucleotides. In some embodiments, surface (3001) may be prepared for bridge amplification of captured oligonucleotide, or other surface amplification technique, for example, as described in International patent publication WO2024/145393, which is incorporated herein by reference. That is, in addition to capture oligonucleotide (3003) there may be additional oligonucleotides attached to surface (3001) to permit surface amplification of the captured oligonucleotides. Surface amplification techniques include, but are not limited to, bridge polymerase chain reaction (bPCR), recombinase-polymerase solid phase amplification (RPA), kinetic exclusion amplification, or the like. Exemplary surface amplification techniques are disclosed in the following references which are incorporated by reference: Adams, U.S. Pat. No. 5,641,658; Boles, U.S. Pat. No. 6,300,070; Mayer, U.S. Pat. Nos. 7,790,418, 7,985,565, 8,652,810, 9,593,328, 9,902,951 and International patent publication WO1998/44151; Ronaghi, U.S. Pat. Nos. 97,773,268, 9,416,415; 7,763,427, 8,426,134, 7,666,598, 9,309,558; or 6,090,592; 6,060,288; 6,787,308; 9,057,097; 9,476,080; 9,476,080; 9,169,513; Adessi et al, Nucleic Acids Research, 28(20): e87 (2000); and the like. In some embodiments, amplification of barcode oligonucleotides may occur primarily on beads (3006), for example, so that bead-amplified barcode oligonucleotides are merely ligated to capture oligonucleotides with the assistance of helper oligonucleotides. In other embodiments, barcode oligonucleotides (including mixed barcode oligonucleotides may be amplified both on beads (for example by way of an SDA) and on surface (3001) by a surface amplification technique, such as, bPCR. As used herein, “surface amplification” means a linear or exponential amplification of a polynucleotide or its complement with at least a portion of the resulting amplicon being covalently attached to the surface.

Unlike the method of International patent publication WO2024/145393, in embodiments of the present method an SDA reaction may be selected so that the quantity of released oligonucleotides is limited only by the amplification process and so that every barcode oligonucleotide released will have a 5′-phosphate, not merely a portion thereof. Thus, in some embodiments, additional surface amplification is optional.

Thus, some embodiments illustrated in FIGS. 3N-3O may proceed as follows: starting with a surface and lawn of capture oligonucleotides barcode oligonucleotides comprising 5′-phosphate groups are released in the presence of helper oligonucleotides and ligase activity on or near the surface, so that at least some barcode oligonucleotides from adjacent beads are ligated to the same capture oligonucleotide to form mixed barcode oligonucleotides. In some embodiments, the helper oligonucleotides may be rendered non-ligatable by the presence of a 3′ blocking group, or a dideoxynucleotide at their 3′ ends.

In some embodiments wherein released barcode oligonucleotide in double stranded form comprise a restriction endonuclease site that is cleaved to expose a particular capture sequence (for example, a polyA terminus for capturing mRNAs) all of the sites except the y the site distal-most from the surface may be inactivated by methylation, as exemplified in International patent publication WO2024/145393.

A method of the embodiment of FIGS. 3N and 3O for making a spatially barcoded surfaces may comprise the following steps: (a) providing a surface comprising capture oligonucleotides attached thereto and a plurality of generator beads disposed thereon, wherein each bead comprises barcode oligonucleotides each comprising a barcode sequence; and (b) generating copies of said barcode oligonucleotides of the generator beads under conditions that copies of the barcode oligonucleotides from at least two different generator beads are captured by said capture oligonucleotides. In some embodiments, the barcode sequence of each different generator bead is different. In some embodiments, conditions under which barcode oligonucleotides are synthesized, released and concatenated into mixed barcode oligonucleotides comprise a concentration of helper oligonucleotides effective for ligating at least a plurality of the generated copies of said barcode oligonucleotides to at least one said capture oligonucleotide. In some embodiments, such plurality of ligated barcode oligonucleotides comprises at least two barcode oligonucleotides from different generator beads. In some embodiments, the method comprises a step to remove unreacted and mis-reacted generated copies, helper oligonucleotides, and like undesired side products from the surface.

FIGS. 4A-4B further illustrate how certain barcoded surfaces of the invention are made and used. As illustrated in the top panel of FIG. 4A, beads with barcode-containing strands are disposed on surface (404) of solid support (400). In some embodiments, surface (404) may be flat and uniform (i.e. non-patterned lithographically, for example) so that beads may be packed into arrays by the flow of a bead-containing solution, for example, when surface (404) is part of a flow chamber or flow cell. In other embodiments, surface (404) may be treated to enhance to formation of stable arrays of a predetermined type, e.g. rectilinear, hexagonal, or the like. In some embodiments, surface (404) may comprise a layer of adhesive, e.g. Performix liquid tape (disclosed by Rodriques et al (cited above)), or like compound, so that after disposition beads remain fixed on surface (404) during subsequent processing steps. In some embodiments, surface (404) may be patterned, e.g. lithographically, to allow the positioning of beads in predetermined or regular arrays. Exemplary regular arrays comprise rectilinear arrays and hexagonal arrays. In some embodiments, regular arrays of features, such as wells or reaction sites, comprise a pitch, or a distance between centers of features on an array. Guidance for manufacturing such arrays is found in Bergo, U.S. Pat. No. 11,131,674; Trau et al, U.S. Pat. No. 10,927,406; and like references, which are incorporated herein by reference. In some embodiments, regular arrays of beads of the invention have pitches in the range of from the equivalents of one bead diameter to four bead diameters. In some embodiments, beads are randomly disposed among the wells or reaction sites of an array. In some embodiments, surface (404) may comprise a predetermined pattern of discrete lithographically formed structures (e.g. wells) or reaction sites which are designed such that a single bead occupies a single well or reaction site. See, for example, Grego et al, Langmuir, 21(11): 4971-4975 (2005), and like references. The occupation of beads in wells or reaction sites may be enhanced by the interaction of chemical groups on the beads with complementary chemical groups on the surface.

Beads may be delivered to surface (404) by a variety of methods including, but not limited to, fluid flow, evaporation, gravity, centrifugation, electrical field, magnetic field, or the like, as exemplified in the following references, which are incorporated herein by reference: Shipway et al, ChemPhysChem, 1:18-52 (2000); Barbee et al, LabChip, 9(22): 3268-3274 (2009); Huang et al, U.S. Pat. No. 9,063,133; Ferguson et al, Anal. Chem., 72:5618-5624 (2000); Shendure et al, Science, 309:1728-1732 (2005); Margulies et al, Nature, 437:376-380 (2005); Trau et al, Science, 272:706-709 (1996); U.S. Pat. Nos. 7,842,649; 7,615,345; 9,436,088; 9,709,559; 10,384,189; and the like. Barbec et al (cited above) describes deposition of streptavidin coated beads to wells formed in a photoresist layer of a multi-layer substrate which includes a gold layer which stabilizes the beads in the wells by a gold-protein interaction. Similar stabilization is available for DNA strands synthesized on beads by a gold-thiol interaction. For example, by using a mixture of nucleoside phosphoramidite and a thiol-modified phosphoramidite (e.g. Glen Research 10-1936) in the last coupling step, a portion of the DNA strands (e.g. 10-25 percent) contain a terminal thiol that may form a bond with the gold layer to stabilize beads in their wells. Photolithographic techniques for forming beads arrays is well known, as exemplified by the following references, which are incorporated herein by reference: Chrisey et al, Nucleic Acids Research, 24(15): 3040-3047 (1996); Drmanac et al, U.S. Pat. No. 8,609,335; Drmanac et al, Science, 327(5961): 78-81 (2009); Brennan et al, U.S. Pat. No. 5,474,796; Kershner et al, Nature Nanotechnology, 4:557-561 (2009); Gopinth et al, ACS Nano, 8(2): 12030-12040 (2014); Thompson et al, J. Micromech. Microeng., 20:115017 (2010); and the like.

After bead array (408) is formed, in some embodiments, it may be encased by gel layer (412), which may be a hydrogel layer, or a degradable hydrogel layer. In some embodiments, gel layer (412) may be thin so that bead layer (408) is just covered, and so that reagents (such as the ligation and/or cleavage reagents) deposited on top, or flowed across the layer, e.g. (414), diffuse rapidly to surface (404) and reach an equilibrium concentration in layer. In some embodiments, thickness (413) of gel layer (412) has a value in the range of from one times to ten times the average diameter of beads (e.g. 430). In other embodiments, thickness (413) of gel layer (412) has a value in the range of from 100-500 percent the average diameter of beads (e.g. 430). In some embodiments, thickness (413) of gel layer (412) has a value in the range of from 10 μm to 500 μm, or in the range of from 20 μm to 200 μm. In some embodiments, the thickness and composition (including porosity) of gel layer (412) is selected so that equilibrium concentrations of reagents are established within an interval selected from the range of from 10 seconds to 5 minutes, or in the range of from 10 seconds to 1 minute. One of ordinary skill in the art recognizes that in some embodiments a trade-off may be necessary between the diffusion rates of selected reagents through gel (412) and the diffusion rate of release barcode strands through gel (412). That is, in some embodiments, on the one hand, it may be desirable to limit diffusion of released barcodes by lowering porosity, while on the other hand, it may be desirable to increase diffusion of ligation reagents by increasing porosity. Thus, a routine design choice confronts one of ordinary skill in the art. In some embodiments, one or more cleavage and/or ligation reagents (e.g. ligase) may be included with gel precursors prior to gelation, so that initiation of ligation occurs upon delivery and diffusion of ligation reagents having smaller molecular sizes (e.g. coenzyme nicotinamide adenine dinucleotide (NAD) for some ligases, e.g. Ampligase). After cleavage of barcoded strands, hybridization of released strands to helper strands and non-released strands, and ligation, and washing to remove unligated released strands and other reaction components, bead array (431) containing mixed barcode strands may be used with (416a) or without (416b) gel layer (412). In embodiment (416b) an extra step of degrading gel (412) is performed prior to applying a tissue sample (435). In some embodiments of (416a—gel intact), gel (412) may be modified but not fully degraded. For example, gel (412) may be modified to increase porosity to facilitate transfer of desired analytes (e.g. mRNA) from tissue sample (435) to bead array (431). In some embodiments, tissue sample (435) may be a microtomed slice comprising a thickness of a few tens of μm, for example, in the range of from 10-50 μm. In some embodiments, tissue samples may be fixed, e.g. FFPE tissues. In some embodiments, such tissue slices may be treated by permeabilizing using conventional techniques so that analytes of interest, e.g. mRNA, are released and more readily transported to the bead array. Villacampa et al, Cell Genomics, 1, 100065 (2021).

Guidance for selecting gels comprising desired properties (including, but not limited to, porosity, gelation speed, degradation characteristics, and the like) for the above uses is provided in the following references, which are incorporated by reference: Lee et al, Prog. Polymer Sci., 37(1): 106-126 (2012); Kharkar et al, Chem. Soc. Rev., 42:7335-7372 (2013); Kharkar et al, Polymer Chem., 6(31): 5565-5574 (2015); Neumann et al, Acta Biomater., 39:1-11 (2016); DeForest et al, Nature Chemistry, 3(12): 925-931(2012); Bowman et al, U.S. Pat. No. 9,631,092; LeValley et al, ACS Appl. Bio. Mater., 3(10): 6944-6958 (2020); Kabb et al, ACS Appl. Mater. Interfaces, 10:16793-16801(2018); Fairbanks et al, Macromolecules, 44:2444-2450 (2011); Fairbanks et al, Adv. Mater., 21(48): 5005-5010 (2009); Sugiura et al, U.S. patent publication US2016/0177030; Shih et al, Biomacromolecules, 13(7): 2003-2012 (2012); and the like. In some embodiments, cleavable linkage 1 comprises a UDG-enzymatically cleavable linkage or an exonuclease V cleavable linkage; cleavable linkage 2 is a photocleavable linkage; and whenever a gel layer (e.g. 412) is employed the gel is an ionic hydrogel, such as alginate or agarose gel, which are degradable by heating or reducing divalent ion concentration. Divalent ions for controlling gelling and degradation of ionic hydrogels, such as agarose or alginate, include, but are not limited to, Mn⁺⁺, Co⁺⁺, Zn⁺⁺, Ni⁺⁺, Cu⁺⁺, Ca⁺⁺ and the like. In other embodiments, cleavable linkage 1 comprises a UDG-based enzymatically cleavable linkage or an endonuclease V cleavable linkage; cleavable linkage 2 is a photocleavable linkage; and whenever a gel layer (e.g. 412) is employed the gel comprises disulfide-containing crosslinking groups which are degradable by treatment with a reducing agent, such as dithiothreitol (DTT), tris(2-carboxyethyl)phosphine (TCEP), tris(3-hydroxypropyl)phosphine (THP), or the like.

In some embodiments, methods for making a spatially barcoded surface may be implemented by the following steps: (a) disposing on a surface a layer of beads comprising at least a first subset of beads wherein each bead comprises first oligonucleotide strands attached, wherein the first oligonucleotide strands each comprise a barcode sequence, and a second subset of beads wherein each bead comprises second oligonucleotide strands cleavably attached thereto, wherein the second oligonucleotide strands each comprise a barcode sequence; (b) embedding the layer of beads on the surface in a gel layer, and (c) cleaving the second oligonucleotide strands under conditions that permit ligation of released second oligonucleotide strands to at least one first oligonucleotide strand of an adjacent bead of the first subset of the layer. In some embodiments, the conditions of cleaving comprise the presence of at least one helper oligonucleotide. In some embodiments, cleaving second oligonucleotide strands comprises photo-cleaving or photo-lysing the second oligonucleotide strands. In some embodiments, a gel layer comprises all of the ligation reagents necessary for ligation, except for a cofactor, so that a ligating step may be implemented by disposing or flowing on the gel a buffer containing the cofactor. In some embodiments, a T4 ligase is employed with an ATP cofactor. In other embodiments, an E. coli ligase is employed with an NAD cofactor.

In some embodiments, the step of disposing beads on a surface may comprise fixing the beads to the surface. In some embodiments, fixing may comprise gluing the beads to the surface. In other embodiments, fixing may comprise beads interacting with micromachined or lithographically formed features on the surface, such as a wells. In still other embodiments, fixing may comprise beads interacting with a reaction site on the surface. In still another embodiment, fixing may comprise beads being embedded in a gel, such as a hydrogel. In some embodiments, fixing beads on a surface comprises more than one of the above methods of fixing the beads. In some embodiments, the layer of beads disposed on the surface is a monolayer. In some embodiments, the gel layer is a degradable hydrogel. In some embodiments, the degradable hydrogel is an agarose gel or an alginate gel.

In some embodiments, the above method further comprises ligating at least one of the released second oligonucleotide strands to at least one of the first oligonucleotide strands of at least one of the adjacent beads to form a mixed barcode strand. In some embodiments, the above method further comprises removing the gel layer from the bead layer. In some embodiments, a gel layer may comprise an agarose gel layer or an alginate gel layer, and removing such gel layer comprises heating the gel layer so that the gel layer melts.

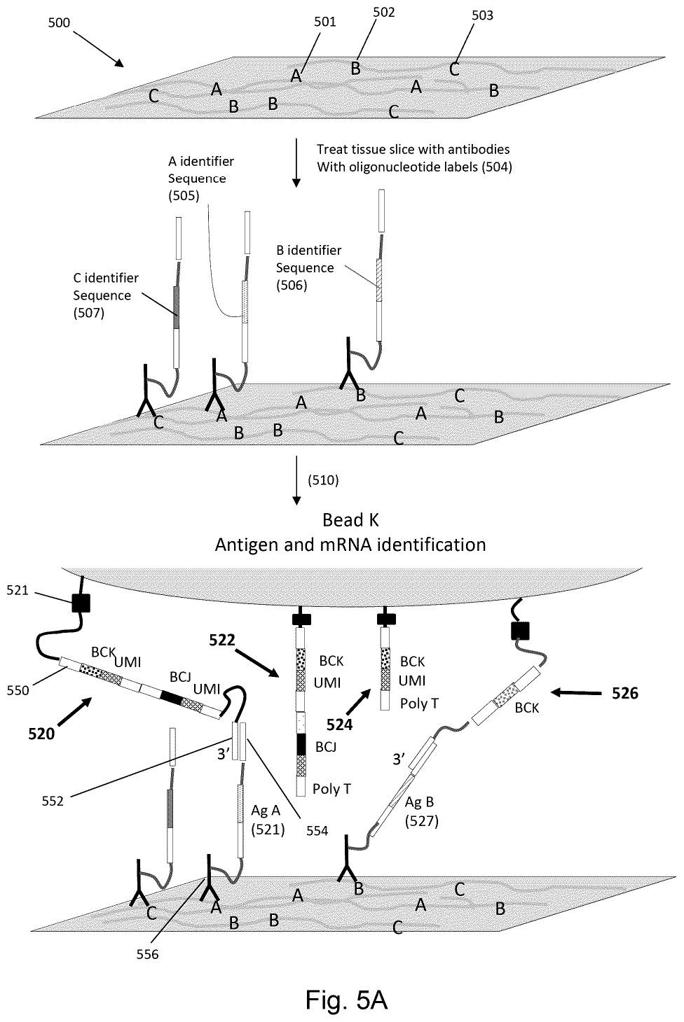

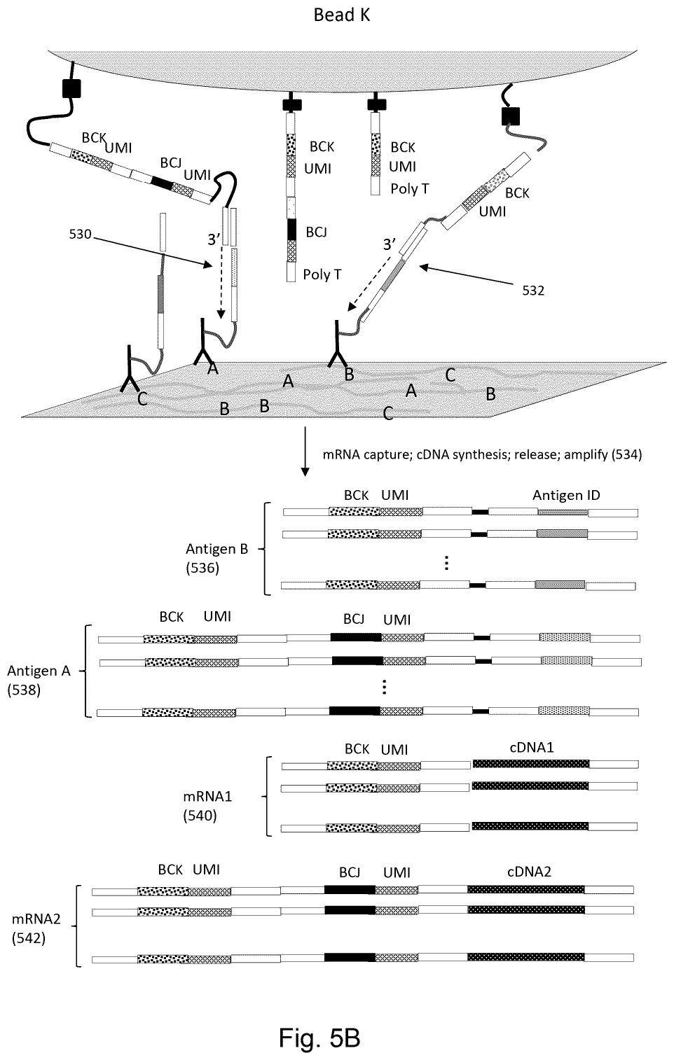

FIGS. 5A-5B illustrate an application of the invention for simultaneously measuring tissue antigens and transcriptome. Tissue slice (500) having antigens A (501), B (502) and C (503) is treated (504) with a mixture of antibodies each comprising an oligonucleotide identification strand (505, 506 and 507) from which the identity of the antigen (A, B or C) to which the antibody binds to can be determined. After incubation and removal, e.g. by washing, non-binding antibodies, tissue slice (500) is placed on a bead array of the invention, bead K of which is shown in the bottom panel of FIG. 5A. Bead K may have attached at least four different strands of which (520, 522, 524 and 526) are shown. Strands not shown include, but are not limited to, mixed barcode strands having secondary barcodes from beads other than bead J and antigen-identification barcode strands hybridized to antigen-identification strands for antigens other than antigen A and antigen B. Bead K comprises oligonucleotide strands for capturing both mRNAs and antibody-identification strands attached to antibodies. Strands (522 and 524) are examples of the former, and strands (520 and 526) are examples of the latter. In addition, strands (520 and 522)) comprise mixed barcode strands from which bead K's position in the bead array may be determined, and strands (522 and 524) comprise single barcode sequences, “BC_{K},” whose positions are determined by the barcode, “BC_{K}” that they share with the other mixed barcode strands on bead K. Mixed barcode strands (520 and 522) illustrate typical elements: cleavable linker (521) proximal to bead K, followed in sequence by a “handle” sequence (550), first barcode sequence “BC_{K}” from bead K, a first UMI sequence, a first capture sequence and a second “handle” sequence (or H1 and H2 sequence), a second barcode sequence “BC_{J},” a second UMI sequence, and capture sequence (552) which is complementary to sequence (554) on oligonucleotide label of antibody (556). After hybridization of antibody capture probes and polyA capture probes to their target sequences (the latter not shown), capture probe strands are extended (e.g. 530 and 532) to produce cDNAs attached to bead K by cleavable linkers (e.g. 521), after which they are released and amplified (534) to produce a collection of amplicons (partially shown in the bottom panel of FIG. 5B, e.g. 536, 538, 540 and 542) which are sequenced. From the sequence data of the mixed barcode strands, the relative positions of beads in the bead array are determined.

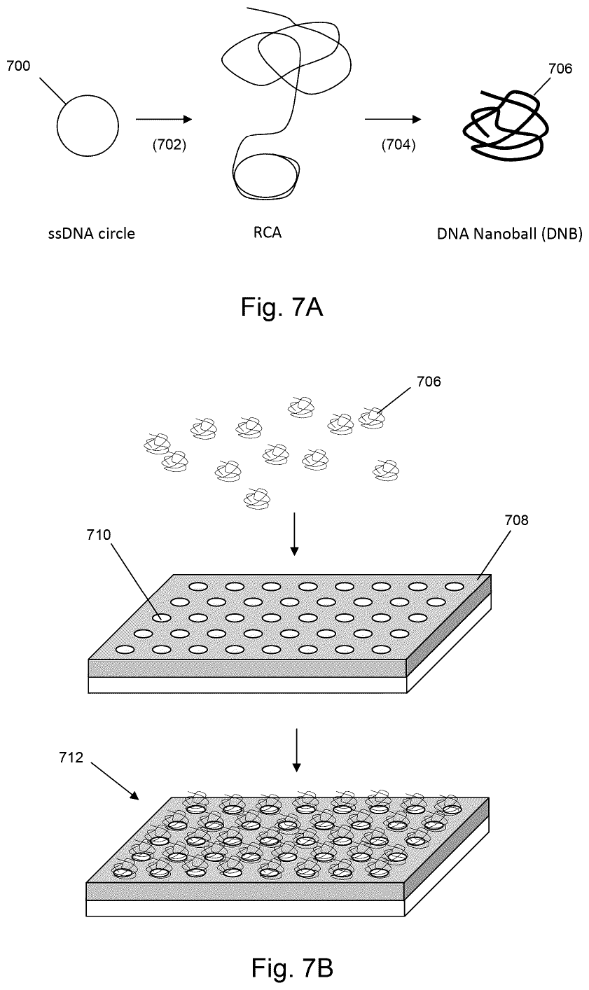

In some embodiments, beads comprise DNA nanoballs which may be produced and disposed on surfaces as taught by Drmanac, e.g. Drmanac et al, U.S. Pat. No. 7,960,104; Drmanac et al, Science, 327:78-81 (2009); and Chen et al, Cell, 185:1777-1792 (2022); Chen et al, U.S. Pat. No. 11,649,489; Chen et al, U.S. patent publication US2023/0175047; and the like, which references are incorporated herein by reference. Briefly, as illustrated in FIG. 7A-7B, a primer (not shown) is hybridized to single stranded DNA circle (700) containing the desired elements and extended (702) by a DNA polymerase to form (704) and amplification product (706), which is referred to herein as a “DNA nanoball,” or DNB. That is, a DNB is a rolling circle amplification (RCA) product or amplicon. In some embodiments, DNBs comprise from 25 to 500 copies of the DNA circle; or from 50 to 300 copies of the DNA circle. In some embodiments, DNBs of the invention may be assembled as described by Chen et al (cited above), with the exception that barcode amplification and polymerase arrest elements are included in the single stranded DNA circle used to generate DNBs.

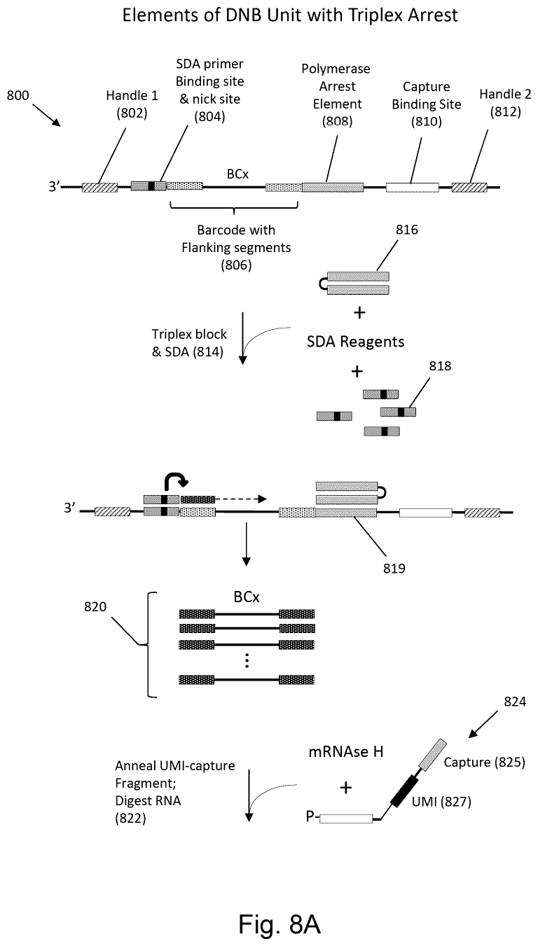

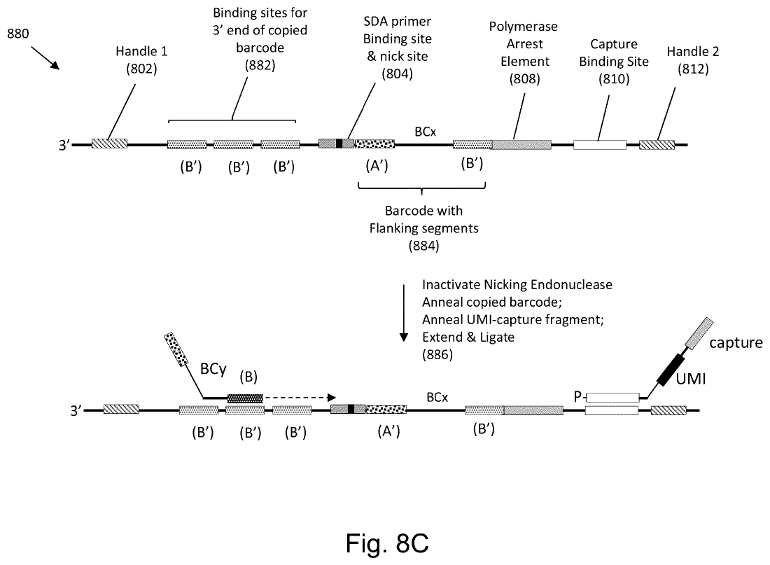

In the embodiment of FIGS. 8A-8C, elements of a DNB are shown which generate copies of its barcode by SDA and triplex arrest. It is well-known that both DNA and RNA triplexes can arrest DNA synthesis by a polymerase, e.g. Samadashwily, “The influence of triplex forming DNA sequences on DNA replication,” Thesis (University of Illinois, 1996); Samadashwily et al, EMBO J., 12(13): 4975-4983 (1993); Samadashwily et al, Gene, 149:127-136 (1994); Krasilnikov et al, Nucleic Acids Research, 25(7): 1339-1346 (1997). It is also known that RNases can digest RNA components of a triplex, e.g. Murray et al, Canadian J. Biochem., 51(4): 436-449 (1973). Thus, in the embodiment SDA is employed to make copies of a barcode in a DNB wherein polymerase synthesis and strand displacement are arrested by an RNA: RNA: DNA triplex formed in the unit. After such replication, the nicking endonuclease may be heat inactivated (e.g. 80° C. for 20 min) after which a UMI-capture fragment is annealed to the DNB unit. RNAse (H or A) is added to digest the RNA components of the triplex, thereby permitting polymerase synthesis through the triplex site. Polymerase, dNTPs, and ligase are then added to complete the joining of the barcode fragment with the UMI-capture fragment, as described by Chen et al (cited above). In FIG. 8A, DNB unit (800) comprises the following elements in a 3′→5′ direction: handle 1 (802) comprises sequences used to construct a DNA circle used to generate the DNB; SDA primer binding site (804) which contains a nicking enzyme recognition site (represented as a black square in the gray rectangle); a barcode sequence with flanking segments (806) (this is the segment replicated by the SDA reaction): a polymerase arrest element (in this embodiment a pyrimidine-rich segment of DNA, e.g. in the range of from 12-24 nucleotides, that forms a triplex with RNA, either one or two strands) (in some embodiments, the polymerase arrest element (819) may comprise the DNA sequence of examples B, C or D of FIG. 19 of Samadashwily's thesis (cited above)); capture binding site (810) to which a UMI-capture fragment is annealed; and handle 2 comprising sequences used to construct a DNA circle. The flanking segments to barcode “BCx” may be the same or different. After RNA strand (816) and SDA primers (818) and associated reagents (e.g., nicking endonuclease and polymerase) are added, copies (820) of barcode, BCx, and its flanking segments are generated. Exemplary polymerases with strand displacement activity for use with the invention include, but are not limited to, E. coli DNA polymerase 1 (exo⁻) Klenow fragment, Φ29, Bst DNA polymerase, Vent (exo⁻), and the like. Exemplary nicking endonucleases for use with the invention include, but are not limited to, Nt.BstNBI, Nt.BspQI, Nt.BspD6I, Nt.Bst91, Nt.BstSEI, Nt.AlwI, and the like. The SDA reaction continues until enough copies of barcode BCx are made so that a portion diffuse to adjacent DNBs and are captured and incorporated into mixed barcode strands. After barcodes are copies, UMI-capture fragments are added under conditions that permit them to anneal to capture binding site (810). UMI-capture fragments may comprise the same structure as the corresponding fragment in Chen et al (cited above). In particular, it comprises a 5′ phosphate group so that it may be ligated to the extended strand from the SDA primer. The capture (825) may be designed to capture natural polynucleotides, e.g. mRNA, or artificial polynucleotides, e.g. antibody labels. Universal molecular indicator (UMI) (827) comprises a random sequence for labeling single molecules. In some embodiments, UMI-capture fragment (824) is annealed to capture binding site (810) after which an RNAse is added to remove the triplex arrest element.

The upper panel of FIG. 8B shows primer (829) annealed to primer binding site “(A)” wherein primer (829) is part of a copied barcode (“BCy”) from an adjacent DNB. Primer (829) of course may anneal to primer binding site “(B)” in which case a mixed barcode strand would not be produced. This inefficiency may be mitigated by using a DNB unit (880) construction as shown in FIG. 8C. In this embodiment, flanking segments (A′) and (B′) (884) are different and a plurality of primer binding sites (882) identical in sequence to (B′) are located in the 3′ direction of SDA primer binding site (804). This construction is more efficient for producing mixed barcode strands than that of FIG. 8A-8B because three of four annealing events by a barcode copy will result in the production of a mixed barcode strand. The additional primer binding sites could also be located in the 5′direction of SDA primer binding site (804) but such a configuration could reduce the efficiency of the strand displacement activity of the polymerase being employed. A plurality of such primer binding sites (882) may be employed. In some embodiments, such plurality is in the range of from 2 to 4. Otherwise, the construction of FIG. 8C operates similarly to that of embodiment of FIGS. 8A-8B, as indicated by steps (886), with the following exception. In some embodiments, the nicking endonuclease of the SDA reaction may be inactivated to prevent strands originating from the (882) binding sites from being cleaved adjacent to the SDA primer. Inactivation may be accomplished by conventional techniques, such as heat inactivation, antibody binding, or the like.

Returning to FIG. 8B, after removal of the triplex block by enzymatic fragmentation of the RNA components (826), primer (829) containing barcode BCy is extended over barcode sequence, BCx (copying its complement), and triplex formation segment (827) to the annealed UMI-capture fragment (824), after which the 3′ end of the extension product is ligated (830) to the 5′ end of the UMI-capture fragment (824). This construct (833) bound to the DNB unit and comprising a mixed barcode strands may then be used to capture target polynucleotides, for example, mRNA (835). Processing captured sequence (835) proceeds by conventional steps to produce construct (837) which may be sequenced to tabulate mixed barcode sequences BCx and BCy as well as a UMI and the identity of the mRNA.

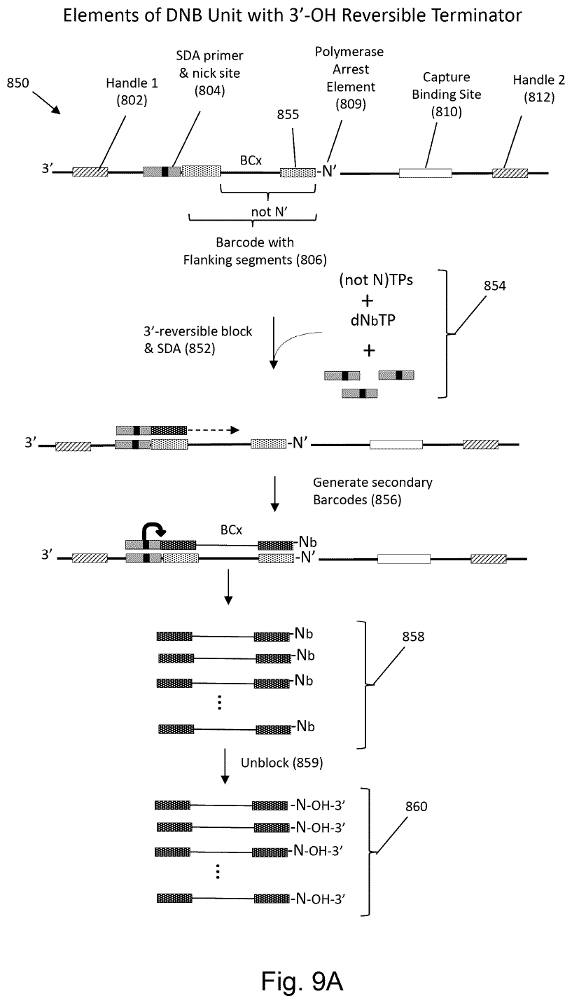

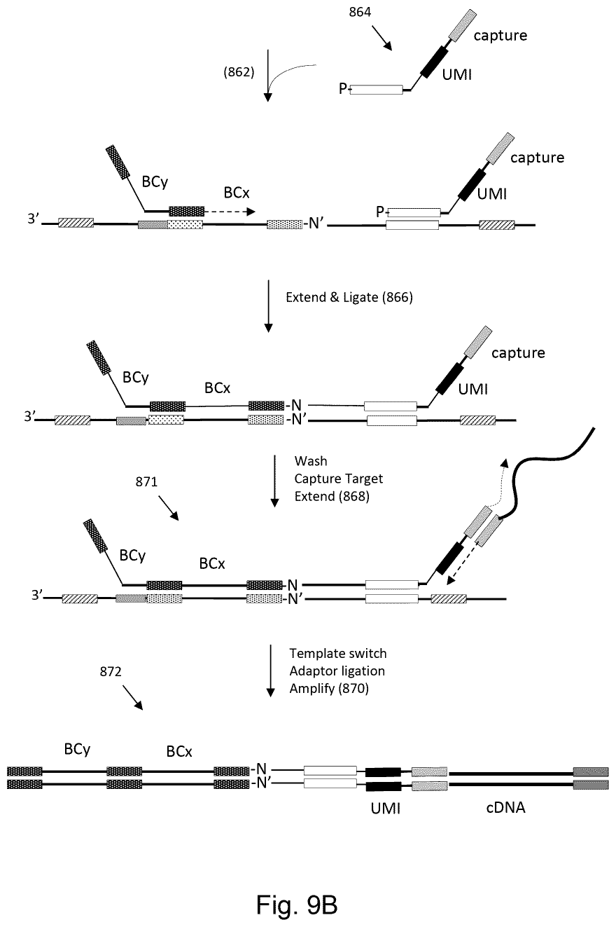

FIGS. 9A-9B illustrate another DNB embodiment in which polymerase activity during SDA is arrested by a reversibly blocked dNTP. In this embodiment, barcodes comprise three out of four kinds of nucleotides, so that a fourth nucleotide can be used comprising a 3′-OH terminator group. That is, for example, in DNB unit (850), segment (806) down stream of SDA primer binding site (804) through segment (855) comprise only three of four nucleotides. The end of segment (855) comprises the fourth nucleotide (shown as N′ in the top panel of FIG. 9A). The dNTP incorporated at that nucleotide comprises a 3′-OH reversible blocking group. More specifically, in various embodiments, segment (806) may comprise A, C, and G and N′ is T; or segment (806) may comprise A, C and T, and N′ is G; or segment (806) may comprise A, G and T, and N′ is C; or segment (806) may comprise G, C and T, and N′ is A. Exemplary reversible 3′-O-blocking groups are well-known and include, but are not limited to, allyl, nitrobenzyl, t-butyldithiomethyl, azidomethyl, and the like. Synthesis and application of such blocking groups are disclosed by Wu, “Analogues for DNA sequencing by synthesis,” Thesis (Columbia University, 2008); U.S. Pat. No. 9,169,510; International patent publication WO 2017/009663; and the like, which references are incorporated herein by reference. In some embodiments, such 3′-O-blocking groups are photoreversible, such as, a 3′-O-nitrobenzyl blocking group, a 3′-O-azidomethyl blocking group, or the like. Upon the addition of dNTPs and SDA reagents (854), an SDA step (856) is performed as in the embodiment of FIGS. 8A-8C to generate secondary barcode-containing strands (858). Strands (858) are then de-blocked, e.g. by irradiation to cleave a photo-labile 3′-O-blocking group, to give strands (860) having free 3′-OH groups. Subsequent steps follow similar to the embodiment of FIGS. 8A-8C: UMI-capture fragment (864) is added (862), after which barcode strands anneal and are extended to the UMI-capture fragments and ligated (866). As with the embodiments of FIGS. 8A-8C, the SDA nicking endonuclease may be inactivated. After ligation, the resulting construct may be employed to capture (868) a target polynucleotide, e.g. mRNA, after which it is extended and processed (870) to produced final product (872) that may be sequenced using conventional methods.

### Applications

After mixed barcode strands are made on a surface in accordance with the invention, oligonucleotide labels, barcodes, genomic fragments, messenger RNAs and similar polynucleotide targets may be captured, copied and sequenced by conventional methods and systems. In some embodiments, the preparation of polynucleotides, e.g. cDNAs, for a sequencing operation takes place after the target templates (e.g. oligonucleotide label, mRNAs, genomic fragments) are released from cells and captured by complementary sequences in the mixed barcode strands. In other embodiments, such target templates may be released from a tissue slice. A releasing step depends on the nature of the target templates. For example, oligonucleotide labels attached to antibodies by a disulfide linkage may be released by a reducing agent (which may also serve as a lysing reagent). mRNAs may be release by treating cells with conventional lysing agents and permeablization agents. Releasing genomic fragments may require lysing and pre-amplification steps. Lysing conditions may vary widely and may be based on the action of heat, detergent, protease, alkaline, or combinations of such factors. The following references provide guidance for selection of lysing reagents, or lysing buffers, for single-cell lysing conditions for mRNA and/or genomic DNA: Thronhill et al, Prenatal Diagnosis, 21:490-497 (2001); Kim et al, Fertility and Sterility, 92:814-818 (2009); Spencer et al, ISME Journal, 10:427-436 (2016); Tamminen et al, Frontiers Microbiol. Methods, 6: article 195 (2015); and the like.

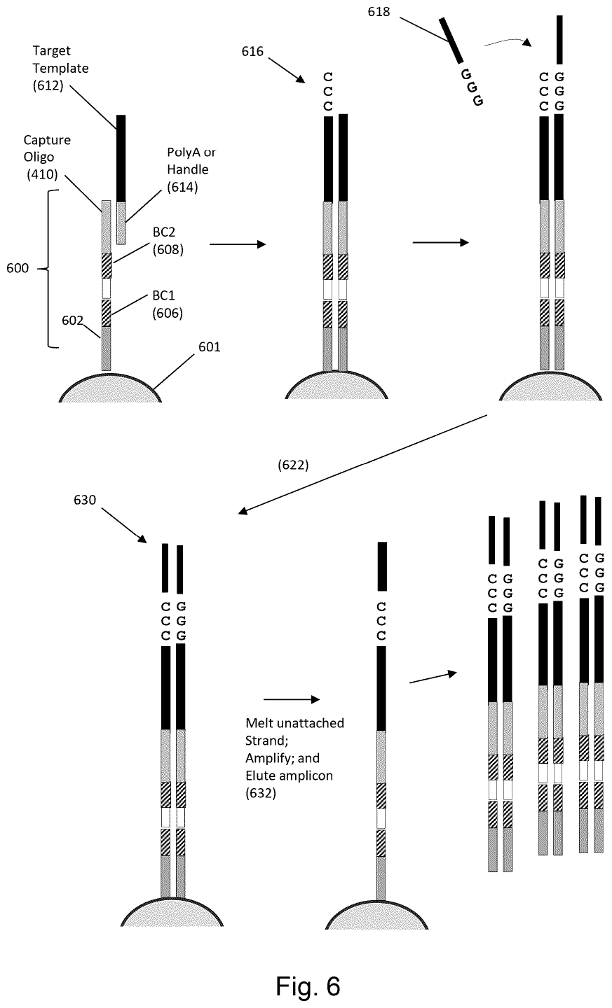

FIG. 6 illustrates a process for capturing target templates and preparing cDNAs for sequencing. One skilled in the art would recognize that the details of the following examples of target mRNA template capture and cDNA synthesis may vary widely depending on the sequencing system employed. In some embodiments, preparation of cDNAs includes a tagmentation step. Guidance for particular embodiments may be found in Picelli et al, Genome Research, 24:2033-2040 (2014); Bose et al, Genome Biology, 16:120 (2015); Hashimshony et al, Genome Biology, 17:77 (2016); Yuan et al, Scientific Reports, 6:33883 (2016); Vickovic et al, Nature Comm., 13182 (2016); U.S. Pat. No. 11,554,370; and like references, which are incorporated herein by reference.

Attached to surface (601) by their 5′ ends are oligonucleotides with the following components: primer binding site P7 (for Illumina sequencers) (602), optional primer binding site R1 (for Illumina paired end sequencing), barcode oligonucleotide (606) (which may be or include a spatial barcode), optional unique molecular identifier (608), and capture oligonucleotide (610), which may be a polyT segment whenever mRNA is to be captured. Target template (612) is captured by the hybridization of polyA segment or sequence handle (614) to capture oligonucleotide (610). After capture, capture oligonucleotide (610) and polyA segment (614) are extended by a polymerase (e.g. Moloney murine leukemia virus (MMLV) reverse transcriptase) that leaves a single stranded polyC tail (616). In some embodiments, template switching oligonucleotide (618) is hybridized thereto and the polyC tail is further extended, as show in (630), e.g. Zhu et al, Biotechniques, 30:892-897 (2001). The unattached strand is melted, the attached strand is amplified, e.g. by a PCR, and eluted for external sequencing (632).

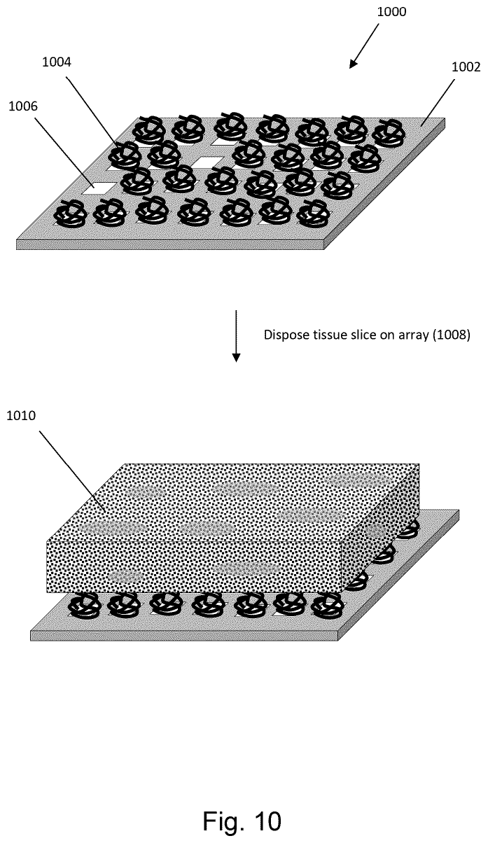

FIG. 10 illustrates a process for capturing target templates and preparing cDNAs for sequencing when capture sequences (such as construct (840) of FIG. 8B or (871) of FIG. 9B) reside on DNBs. DNB array (1000) is provided comprising surface (1002) with reaction sites (1006) to which DNBs (1004) preferentially bind. Each DNB comprises a plurality of capture constructs, such as (840) or (871) (not shown in FIG. 10) which are capable of capturing by hybridization desired biomolecules such as mRNA. After conventional preparation (e.g. permeabilization), tissue slice (1010) is disposed on DNB array (1000) as described by Chen et al (cited above) under conditions that permit capture of the desired biomolecules and their processing to produce final products, such as (839) or (872).

### Kits

The present invention comprises kits for performing methods of the invention. Generally, a kit may be any delivery system for delivering materials or reagents for carrying out a method of the invention. Such delivery systems include systems that allow for the storage, transport, or delivery of reagents and/or hardware components (e.g., a substrate comprising a surface comprising beads comprising mixed barcode strands in the appropriate containers) and/or supporting materials (e.g., buffers, written instructions for performing an assay, instructions and/or software for determining mixed barcode locations from sequence data, etc.) from one location to another. For example, kits may include one or more enclosures (e.g., boxes) containing the relevant reagents, articles of manufacture, such as a bead array, and/or supporting materials. Such contents may be delivered to the intended recipient together or separately. In some embodiments, a kit for using bead arrays of the invention may comprise one or more of the following: a solid support comprising a planar surface comprising a bead array thereon, wherein beads of the array comprise oligonucleotide strands comprising mixed barcode strands. In some embodiments, such bead array is a closely packed array. In some embodiments, such bead array comprises EXPAR beads (or SDA beads). In some embodiments, the above kit may further comprise instructions and/or software for determining relative bead locations from the DNA sequences of mixed barcode strands in the bead array. In some embodiments, such instructions and/or software for determining relative bead locations further includes bead array image data. In some embodiments, such software for computing relative bead locations may be delivered as an electronic file, either on storage media or as a download from a website.

In some embodiments, a kit for methods of making bead arrays of the invention may comprise a solid support comprising a surface comprising a lithographically patterned surface of reaction sites or wells configured to accept beads disposed thereon. In some embodiments, such kits may further comprise beads comprising barcode sequences, wherein different beads comprise different barcode sequences, or wherein each bead comprises a unique barcode sequence. In some embodiments, such kits further comprise ligation reagents. In some embodiments, such kits further comprise cleavage reagents. In some embodiments, such kits further comprise a diffusion inhibitor.

In some embodiments, a kit for methods of making bead arrays of the invention may comprise one or more EXPAR beads (or SDA beads). In some embodiments, each EXPAR bead comprises one or more oligonucleotide strands each comprising the same unique barcode sequence and each configured for performing EXPAR to produce copies of a complementary strand thereof. In some embodiments, such kits further include a nicking enzyme and a DNA polymerase.

### Further Embodiments of the Invention

The following are embodiments of the invention: A method of making a spatially barcoded surface comprising: (a) disposing on a surface a layer of beads comprising at least a subset of beads wherein each bead comprises first oligonucleotide strands and second oligonucleotide strands, wherein the first and second oligonucleotide strands each comprise a barcode sequence such that first and second oligonucleotides of of the same bead comprise the same barcode sequence; and (b) cleaving the second oligonucleotide strands under conditions that permit ligation of released second oligonucleotide strands to first oligonucleotide strands of adjacent beads of the layer. The above method further comprising ligating at least one of said released second oligonucleotide strands to at least one of said first oligonucleotide strands of at least one of said adjacent beads to form a mixed barcode strand. The above method further comprising capturing nucleic acids from a sample with said mixed barcode strands and extending said mixed barcode strands to form mixed barcoded cDNAs. The above method further comprising determining relative positions of said barcodes on said surface from the sequences of said barcodes of said mixed barcoded cDNAs. The above method wherein said first oligonucleotide strands are cleavably attached to said beads, further comprising cleaving and sequencing said mixed barcode cDNAs. The above method further comprising imaging said layer of beads on said surface to obtain bead image data and determining positions of said barcodes on said surface from the sequences of said barcodes of said mixed barcoded cDNAs and the bead image data. The above method wherein said beads are disposed randomly on said surface. The above method wherein said layer of beads on said surface is closely packed. The above method wherein said barcode on each of said beads is unique. The above method wherein said barcode sequences of oligonucleotide strands attached to different beads are different. The above method wherein said surface comprises a regular array of wells or reaction sites each configured to retain a single bead. The above method wherein said beads are disposed randomly among said wells or reaction sites. The above method wherein said wells or reaction sites of said regular array have a pitch in the range of from one bead diameter to three bead diameters.

The following are further embodiments of the invention: A method for measuring tissue-wide expression of biomolecules, the method comprising: (a) providing a bead array comprising beads comprising mixed barcode strands; (b) disposing a tissue slice on the bead array; (c) capturing target nucleic acids released from tissue slice by mixed barcode strands; (d) synthesizing cDNAs having mixed barcode strands from the captured target nucleic acids; (e) sequencing the cDNAs: and (f) determining relative positions of the captured target nucleic acids in the bead array from sequences of the mixed barcode strands. The above method wherein said bead array comprises bead image data and said determining positions of said captured target nucleic acids is based on said sequences of said mixed barcode strands and the bead image data.

The following are further embodiments of the invention: A kit for measuring tissue-wide expression of biomolecules, the kit comprising a solid support comprising a surface comprising a layer of beads comprising mixed barcode strands. The above kit further comprising instructions or software for determining positions of said biomolecules based on sequences of said mixed barcode strands. The above kit further comprising bead image data for said layer of beads. The above kit further comprising instructions or software for determining positions of said biomolecules based on sequences of said mixed barcode strands and said bead image data.

The following are further embodiments of the invention: An article of manufacture for making oligonucleotides each comprising the same barcode sequence, the article of manufacture comprising one or more EXPAR beads. The above article of manufacture wherein each of said EXPAR beads comprises oligonucleotide strands attached thereto wherein each oligonucleotide strand comprises a nicking endonuclease binding site and a unique barcode sequence.

The following are further embodiments of the invention: An article of manufacture for measuring spatial distributions of biomolecules, the article of manufacture comprising a solid support comprising a surface comprising a layer of beads comprising mixed barcode strands. The above article of manufacture wherein said layer of beads comprises EXPAR beads.

The following are further embodiments of the invention: A product for measuring spatial distributions of biomolecules made by a process comprising: (a) disposing on a surface a layer of beads comprising at least a subset of beads wherein each bead comprises first oligonucleotide strands and second oligonucleotide strands, wherein the first and second oligonucleotide strands each comprise a barcode sequence such that first and second oligonucleotides of of the same bead comprise the same barcode sequence; (b) cleaving the second oligonucleotide strands under conditions that permit ligation of released second oligonucleotide strands to first oligonucleotide strands of adjacent beads of the layer; and (c) ligating at least one of the released second oligonucleotide strands to at least one of the first oligonucleotide strands of at least one of said adjacent beads to form a mixed barcode strand. The above product by process wherein said beads of said layer are fixed to said surface.

The following are further embodiments of the invention: A product for measuring spatial distributions of biomolecules made by a process comprising: (a) disposing on a surface a layer of beads comprising at least a first subset of beads wherein each bead comprises first oligonucleotide strands attached, wherein the first oligonucleotide strands each comprise a barcode sequence, and a second subset of beads wherein each bead comprises second oligonucleotide strands cleavably attached, wherein the second oligonucleotide strands each comprise a barcode sequence; (b) cleaving the second oligonucleotide strands under conditions that permit ligation of released second oligonucleotide strands to at least one first oligonucleotide strand of an adjacent bead of the first subset of the layer; and (c) ligating at least one of the released second oligonucleotide strands to at least one of the first oligonucleotide strands of at least one of the adjacent beads to form a mixed barcode strand. The above product by process wherein said beads of said layer are fixed to said surface. The above product by process further comprising embedding said layer of beads in a gel layer.

The following are further embodiments of the invention: An article of manufacture for constructing an array of beads comprising mixed barcode strands, the article of manufacture comprising a solid support comprising a surface comprising a layer of beads comprising generator beads and acceptor beads. The above article of manufacture wherein each of said generator beads comprises an optical label indicative of a secondary barcode of said generator bead. The above article of manufacture wherein each generator bead comprises an effective diffusion area and wherein each acceptor bead of said array resides within the effective diffusion area of at least two generator beads. The above article of manufacture wherein said generator beads comprise EXPAR beads.

The following are further embodiments of the invention: A method of making a spatially barcoded surface comprising: (a) disposing on a surface a layer of beads comprising oligonucleotide strands, wherein the oligonucleotide strands of each comprise a barcode sequence; (b) releasing the oligonucleotide strands under conditions that permit the released oligonucleotide strands to be concatenated with at least one other oligonucleotide strand of an adjacent bead to form a mixed barcode strand. The above method wherein said releasing comprises replicating said first oligonucleotide strands. The above method wherein said bead is a DNA nanoball. The above method wherein said replicating comprises strand displacement amplification. The above method wherein said mixed barcode strand comprises a capture strand complementary to a portion of a target polynucleotide.

While the present invention has been described with reference to several particular example embodiments, those skilled in the art will recognize that many changes may be made thereto without departing from the spirit and scope of the present invention. The present invention is applicable to a variety of biomolecular measurements and other subject matter, in addition to those discussed above.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immuology, 6^{th} edition (Saunders, 2007); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Sambrook, Fritsche and Maniatis. “Molecular Cloning A laboratory Manual” 2nd Ed. Cold Spring Harbor Press (1989); and the like.

“Barcode” means a molecular label or identifier. In some embodiments, barcodes comprise oligonucleotides. In some embodiments, oligonucleotide barcodes comprise one or more segments that encode by nucleotide sequences information about a biomolecule, such as its identity, location, or relative location with respect to other barcodes. Such oligonucleotide barcodes are “spatial barcodes” in that they encode information about locations or relative locations of biomolecules. In some embodiments, a barcode is a molecule attached to an analyte or a segment of an analyte (for example, in the case of polynucleotide barcodes and analytes) which may be used to identify the analyte, which may or may not be a biomolecule. In some embodiments, there may be a one-to-one correspondence between spatial barcodes comprising different nucleotide sequences and different areas on a surface; that is, each different area has a different and unique barcode. In some embodiments, the identity of a spatial barcode is determinable, for example, by sequencing whenever a spatial barcode is a polynucleotide. In some embodiments, a spatial barcode is an oligonucleotide. In some embodiments, oligonucleotide spatial barcodes comprise random sequence oligonucleotides. Random sequence oligonucleotides are typically synthesized by a “split and mix” synthesis techniques, for example, as described in the following references that are incorporated herein by reference: Church, U.S. Pat. No. 4,942,124; Godron et al, International patent publication WO2020/120442; Seelig et al, U.S. patent publication 2016/0138086; and the like. Sometimes random oligonucleotides are represented as “NNN . . . N.” In some embodiments, random sequence oligonucleotides used as barcodes have a size in the range of from 8 to 30 nucleotides, or from 8 to 20 nucleotides. In some embodiments, the term “barcode” includes composite barcodes; that is, an oligonucleotide segment that comprises sub-segments that identify different objects. For example, a first segment of a composite barcode may identify a particular area on a surface and a second segment of a composite barcode may identify a particular molecule (for example, a so-called “unique molecular identifier” or UMI). In some embodiments, a “surface” comprises beads disposed on a planar surface. In some embodiments, such beads on a planar surface are closely packed.

“Biomolecule” means a molecule typically, but not always, derived from a living organism which can be directly or indirectly associated with, or attached to, a barcode. In some embodiments, biomolecules comprise proteins or nucleic acids. In some embodiments, biomolecules comprise proteins. In other embodiments, biomolecules comprise nucleic acids. In some embodiments, biomolecules comprise RNAs or DNAs. In some embodiments, biomolecules comprise messenger RNAs (mRNAs). In some embodiments, biomolecules comprise complementary DNAs (cDNAs). cDNAs may be derived from mRNAs or other polynucleotides.

“Cleavable linkage” or “cleavable nucleotide” means any of wide variety of cleavable linkages, or more particularly, cleavable nucleotides, may be used with embodiments of the invention. As used herein, the term “cleavable site” or “cleavable linkage” refers to a nucleotide or backbone linkage of a single stranded nucleic acid sequence that can be excised or cleaved under predetermined conditions, thereby separating the single stranded nucleic acid sequence into two parts. In some embodiments, a step of cleaving a cleavable nucleotide or a cleavable linkage leaves a free 3′-hydroxyl on a cleaved strand, thereby, for example permitting the cleaved strand to be extended by a polymerase. In other embodiments, a cleavable nucleotide or cleavable linkage leaves a 5′-phosphate group on a cleaved strand, thereby permitting the strand to ligated to another strand having a free 3′-hydroxyl. Cleaving steps may be carried out chemically, thermally, enzymatically or by light-based cleavage. Sometimes the term “releasing” may be used in reference to cleaving an oligonucleotide strand, for example, by a releasing reagent or agent, which may be one or more of those listed above. In some embodiments, cleavable nucleotides may be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavage by glycosylases and/or endonucleases may require a double stranded DNA substrate. Methods synthesizing and cleaving nucleic acids containing chemically cleavable, thermally cleavable, and photo-labile groups are described for example, in U.S. Pat. No. 5,700,642, which is incorporated herein by reference. Further cleavable linkages are disclosed in the following references: Pon, R., Methods Mol. Biol. 20:465-496 (1993); Verma et al., Ann. Rev. Biochem. 67:99-134 (1998); U.S. Pat. Nos. 5,739,386, 5,700,642 and 5,830,655; and U.S. Patent Publication Nos. 2003/0186226 and 2004/0106728, Urdea et al, U.S. Pat. No. 5,367,066; Creton, U.S. Pat. No. 11,359,221, which are incorporated herein by reference. Synthesis and cleavage conditions of chemically cleavable oligonucleotides are described in U.S. Pat. Nos. 5,700,642 and 5,830,655. Phosphorothioate internucleotide linkage may be selectively cleaved under mild oxidative conditions. Selective cleavage of the phosphoramidate bond may be carried out under mild acid conditions, such as 80% acetic acid. Selective cleavage of ribose may be carried out by treatment with dilute ammonium hydroxide. In another embodiment, a cleavable linking moiety may be an amino linker. The resulting oligonucleotides bound to the linker via a phosphoramidite linkage may be cleaved with 80% acetic acid yielding a 3′-phosphorylated oligonucleotide. In some embodiments, the cleavable linking moiety may be a photocleavable linker, such as an ortho-nitrobenzyl photocleavable linker. Synthesis and cleavage conditions of photolabile oligonucleotides on solid supports are described, for example, in Venkatesan et al., J. Org. Chem. 61:525-529 (1996), Kahl et al., J. Org. Chem. 64:507-510 (1999), Kahl et al., J. Org. Chem. 63:4870-4871 (1998), Greenberg et al., J. Org. Chem. 59:746-753 (1994), Holmes et al., J. Org. Chem. 62:2370-2380 (1997), and U.S. Pat. No. 5,739,386. Ortho-nitrobenzyl-based linkers, such as hydroxymethyl, hydroxyethyl, and Fmoc-aminoethyl carboxylic acid linkers, may also be obtained commercially. In some embodiments, ribonucleotides may be employed as cleavable nucleotides, wherein a cleavage step may be implemented using a ribonuclease, such as RNase H. In other embodiments, cleavage steps may be carried out by treatment with a nickase.

“DNA nanoballs,” “DNBs,” or “RCA amplicons” are generally concatemers comprising multiple copies of a single stranded DNA circle. In certain aspects, DNBs comprise repeating monomeric units, each monomeric unit comprising one or more functional elements, such as primer binding sites, hybridization sites, SDA primer binding sites, secondary barcode capture sites, and the like. In one aspect, rolling circle amplification (RCA) is used to create concatemers of the invention. Guidance for selecting conditions and reagents for RCA reactions is available in many references available to those of ordinary skill, including U.S. Pat. Nos. 5,426,180; 5,854,033; 6,143,495; and 5,871,921, each of which is hereby incorporated by reference in its entirety for all purposes and in particular for all teachings related to generating concatemers using RCA or other methods. Generally, RCA reaction components include single stranded DNA circles, one or more primers that anneal to DNA circles, a DNA polymerase having strand displacement activity to extend the 3′ ends of primers annealed to DNA circles, nucleoside triphosphates, and a conventional polymerase reaction buffer. Such components are combined under conditions that permit primers to anneal to DNA circle. Extension of these primers by the DNA polymerase forms concatemers of DNA circle complements. In some embodiments, nucleic acid templates of the invention are double stranded circles that are denatured to form single stranded circles that can be used in RCA reactions. Methods for forming DNBs of the invention are described in Published Patent Application Nos. WO2007120208, WO2006073504, WO2007133831, and US2007099208, all of which are incorporated herein by reference in their entirety for all purposes and in particular for all teachings related to forming DNBs.

“Kit” refers to any delivery system for delivering materials or reagents for carrying out a method of the invention. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes. In one aspect of the present invention, kits also include in one aspect circularizing adaptors for enumerating particular DNA fragments, such as selected regions of the ErbB2 gene, or the like. Such kits also include one or more type IIs restriction endonucleases, such as double cleavage type IIs restriction endonucleases. Such kits further include reagents for internal and external standards, such as a second circularizing adaptor for an internal standard fragment indigenous to a specimen, and/or such as a known DNA fragment for an external standard that has a known concentration (and therefore, a known number in a predetermined reaction volume). In another aspect, kits also include padlock probes specific for selected regions of particular genes as described above, probe extension reagents, probe ligation reagents, one or more nucleases, and components for capture, primer extension, and extension product amplification. In still another aspect, kits also include ligation probes comprising a first component and a second component, ligation reagents, reagents for amplifying and capturing ligation products.

“Ligation” means to form a covalent bond or linkage between the termini of two or more nucleic acids, e.g. oligonucleotides and/or polynucleotides, usually in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27:875-881 (1999); Higgins et al, Methods in Enzymology, 68:50-71 (1979); Engler et al, The Enzymes, 15:3-29 (1982); and Namsaraev, U.S. patent publication 2004/0110213. A typical reaction buffer (10×) of a commercially available ligase (Ampligase, from Epicentre) which may be used with the invention comprises 200 mM Tris-HCl (pH 8.3), 250 mM KCl, 100 mM MgCl₂, 5 mM NAD, and 0.1% Triton X-100.

“Polynucleotide” or “oligonucleotide” are used interchangeably and each mean a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moities, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 14 to 36 nucleotides.

What is claimed is:

1. A method of making a spatially barcoded surface comprising:
disposing on a surface a layer of beads comprising at least a first subset of beads wherein each bead comprises first oligonucleotide strands attached, wherein the first oligonucleotide strands each comprise a barcode sequence, and a second subset of beads wherein each bead comprises second oligonucleotide strands cleavably attached, wherein the second oligonucleotide strands each comprise a barcode sequence; and
cleaving the second oligonucleotide strands under conditions that permit ligation of released second oligonucleotide strands to at least one first oligonucleotide strand of an adjacent bead of the first subset of the layer.

2. The method of claim 1 further comprising ligating at least one of said released second oligonucleotide strands to at least one of said first oligonucleotide strands of at least one of said adjacent beads to form a mixed barcode strand.

3. The method of claim 2 further comprising capturing nucleic acids from a sample with said mixed barcode strands and extending said mixed barcode strands to form mixed barcoded cDNAs.

4. The method of claim 3 further comprising determining relative positions of said barcodes on said surface from the sequences of said barcodes of said mixed barcoded CDNAs.

5. The method of claim 3, wherein said first oligonucleotide strands are cleavably attached to said beads, further comprising cleaving and sequencing said mixed barcode cDNAs.

6. The method of claim 3 further comprising imaging said layer of beads on said surface to obtain bead image data and determining positions of said barcodes on said surface from the sequences of said barcodes of said mixed barcoded cDNAs and the bead image data.

7. The method of claim 1 wherein said beads are disposed randomly on said surface.

8. The method of claim 1 wherein said layer of beads on said surface is closely packed.

9. The method of claim 1 wherein said barcode on each of said beads is unique.

10. The method of claim 1 wherein said barcode sequences of oligonucleotide strands attached to different beads are different.

11. The method of claim 1 wherein said surface comprises a regular array of wells or reaction sites each configured to retain a single bead.

12. The method of claim 11 wherein said beads are disposed randomly among said wells or reaction sites.

13. The method of claim 11 wherein said wells or reaction sites of said regular array have a pitch in the range of from one bead diameter to three bead diameters.

14. The method of claim 11, wherein said second oligonucleotide strands are capable of being amplified and released, further comprising amplifying and releasing the second oligonucleotide strands under conditions that permit ligation of released second oligonucleotide strands to first oligonucleotide strands of at least one adjacent bead of said first subset of beads.

15. The method of claim 14 wherein said second oligonucleotide strands are amplified and released by a strand displacement amplification reaction.

16. The method of claim 15 wherein said strand displacement reaction is an exponential amplification reaction (EXPAR).

17. The method of claim 15 further comprising ligating said released second oligonucleotide strands to said first oligonucleotide strands of said adjacent beads to form mixed barcode strands.

18. A method of making a spatially barcoded surface, comprising:
providing a surface comprising capture oligonucleotides attached thereto and a plurality of generator beads disposed thereon, wherein each bead comprises barcode oligonucleotides each comprising a barcode sequence;
generating copies of the barcode oligonucleotides of the generator beads under conditions that copies of the barcode oligonucleotides from at least two different generator beads are ligated to the same capture oligonucleotide.

19. The method of claim 18 wherein said conditions comprise a concentration of helper oligonucleotides effective for ligating at least a plurality of said generated copies of said barcode oligonucleotides to at least one said capture oligonucleotide.

20. The method of claim 19 wherein said generating comprises performing a strand displacement amplification reaction.
